(19) United States
(12) Patent Application Publication
(10) Pub. No.: US 2025/0113116 A1
(43) Pub. Date: Apr. 3, 2025

(54) DETECTION DEVICE

(71) Applicant: Japan Display Inc., Tokyo (JP)

(72) Inventor: Gen KOIDE, Tokyo (JP)

(21) Appl. No.: 18/958,336

(22) Filed: Nov. 25, 2024

Related U.S. Application Data
(63) Continuation of application No. PCT/JP2023/019781, filed on May 26, 2023.

(30) Foreign Application Priority Data
May 27, 2022 (JP) 2022-086830

(51) Int. Cl. H04N 25/772; H04N 25/78; H10K 39/34; H10K 39/38

(52) U.S. Cl. CPC H04N 25/772; H04N 25/78; H10K 39/34; H10K 39/38

(57) ABSTRACT

According to an aspect, a detection device includes: an optical sensor; a light source configured to emit light to the optical sensor; a detection signal amplifying circuit configured to convert a variation of current supplied from the optical sensor into a variation of voltage; and an analog-to-digital (A/D) conversion circuit configured to convert an output voltage signal after being converted into the voltage variation into a digital detection value. The A/D conversion circuit is configured to limit the detection value to a maximum digital gradation value or a minimum digital gradation value when the light source is off.

### CROSS-REFERENCE TO RELATED APPLICATION

This application claims the benefit of priority from Japanese Patent Application No. 2022-086830 filed on May 27, 2022 and International Patent Application No. PCT/JP2023/019781 filed on May 26, 2023, the entire contents of which are incorporated herein by reference.

### BACKGROUND

### 1. Technical Field

What is disclosed herein relates to a detection device.

### 2. Description of the Related Art

Detection devices are known that emit light into a body through the skin thereof and acquire an oxygen saturation level in blood (hereinafter, called “blood oxygen saturation level (SpO₂)”) based on transcutaneous data acquired by detecting light transmitted through or reflected by arteries. The blood oxygen saturation level (SpO₂) refers to a ratio of an amount of oxygen actually bound to hemoglobin to the total amount of oxygen under the assumption that the oxygen is bound to all the hemoglobin in the blood. When acquiring the blood oxygen saturation level (SpO₂), for example, a pulse wave acquired by red light and a pulse wave acquired by infrared light are used (refer to Japanese Patent Application Laid-open Publication No. 2019-180861, for example).

In a configuration that uses an optical sensor such as an organic photodiode (OPD) to acquire the pulse wave, a variation of current supplied from the optical sensor is converted into a variation of voltage and the converted voltage is amplified, and the output voltage signal after the voltage conversion is converted into a digital value, thus acquiring the pulse wave. In such a configuration, the variation component of the pulse wave is small with respect to the full scale range of the digital value, and sufficient accuracy may not be obtained.

For the foregoing reasons, there is a need for a detection device capable of improving the accuracy of detection of waveforms of pulse waves.

### SUMMARY

According to an aspect, a detection device includes: an optical sensor; a light source configured to emit light to the optical sensor; a detection signal amplifying circuit configured to convert a variation of current supplied from the optical sensor into a variation of voltage; and an analog-to-digital (A/D) conversion circuit configured to convert an output voltage signal after being converted into the voltage variation into a digital detection value. The A/D conversion circuit is configured to limit the detection value to a maximum digital gradation value or a minimum digital gradation value when the light source is off.

According to an aspect, a detection device includes: an optical sensor; a light source configured to emit light to the optical sensor; a detection signal amplifying circuit configured to convert a variation of current supplied from the optical sensor into a variation of voltage; and an output circuit configured to convert an output voltage signal after being converted into the voltage variation into a detection value. The output circuit is configured to limit the detection value to a maximum detection value or a minimum detection value when the light source is off.

### BRIEF DESCRIPTION OF THE DRAWINGS

FIG. 1 is a plan view illustrating a detection device according to a first embodiment;

FIG. 2 is a block diagram illustrating a configuration example of the detection device according to the first embodiment;

FIG. 3 is a circuit diagram illustrating the detection device according to the first embodiment;

FIG. 4 is a circuit diagram illustrating a configuration example of an analog front-end (AFE) circuit according to the first embodiment;

FIG. 5 is a schematic partial sectional view of an optical sensor according to the first embodiment;

FIG. 6 is a timing waveform diagram illustrating an operation example of a detection device according to a comparative example for the first embodiment;

FIG. 7 is an enlarged diagram of a readout period for each optical sensor in the timing waveform diagram illustrated in FIG. 6;

FIG. 8 is a diagram illustrating a correspondence relation between a detection value of the AFE circuit and a received light intensity of the optical sensor in the operation example of the detection device according to the comparative example for the first embodiment;

FIG. 9 is a timing waveform diagram illustrating an operation example of the detection device according to the first embodiment;

FIG. 10 is an enlarged diagram of the readout period for each optical sensor in the timing waveform diagram illustrated in FIG. 9;

FIG. 11 is a first diagram illustrating the correspondence relation between the detection value of the AFE circuit and the received light intensity of the optical sensor in a first operation example of the detection device according to the first embodiment;

FIG. 12 is a second diagram illustrating the correspondence relation between the detection value of the AFE circuit and the received light intensity of the optical sensor in the first operation example of the detection device according to the first embodiment;

FIG. 13 is a first diagram illustrating the correspondence relation between the detection value of the AFE circuit and the received light intensity of the optical sensor in a second operation example of the detection device according to the first embodiment;

FIG. 14 is a second diagram illustrating the correspondence relation between the detection value of the AFE circuit and the received light intensity of the optical sensor in the second operation example of the detection device according to the first embodiment;

FIG. 15 is a circuit diagram illustrating a configuration example of an AFE circuit according to a modification of the first embodiment;

FIG. 16 is a timing waveform diagram illustrating an operation example of the detection device according to the modification of the first embodiment;

FIG. 17 is an enlarged diagram of the readout period for each optical sensor in the timing waveform diagram illustrated in FIG. 16;

FIG. 18 is a diagram illustrating the correspondence relation between the detection value of the AGE circuit and the received light intensity of the optical sensor in the operation example of the detection device according to the modification of the first embodiment;

FIG. 19 is a circuit diagram illustrating the detection device according to a second embodiment;

FIG. 20 is a circuit diagram illustrating a configuration example of the AFE circuit according to the second embodiment;

FIG. 21 is a diagram illustrating the correspondence relation between the detection value of the AFE circuit and the received light intensity of the optical sensor in an operation example of a detection device according to a comparative example for the second embodiment;

FIG. 22 is a timing waveform diagram illustrating an operation example of the detection device according to the second embodiment;

FIG. 23 is an enlarged diagram of the readout period for each optical sensor in the timing waveform diagram illustrated in FIG. 22;

FIG. 24 is a first diagram illustrating the correspondence relation between the detection value of the AFE circuit and the received light intensity of the optical sensor in the operation example of the detection device according to the second embodiment;

FIG. 25 is a second diagram illustrating the correspondence relation between the detection value of the AFE circuit and the received light intensity of the optical sensor in the operation example of the detection device according to the second embodiment;

FIG. 26 is a circuit diagram illustrating a configuration example of the AFE circuit according to a modification of the second embodiment;

FIG. 27 is a timing waveform diagram illustrating an operation example of the detection device according to the modification of the second embodiment;

FIG. 28 is an enlarged diagram of the readout period for each optical sensor in the timing waveform diagram illustrated in FIG. 27; and

FIG. 29 is a diagram illustrating the correspondence relation between the detection value of the AFE circuit and the received light intensity of the optical sensor in the operation example of the detection device according to the modification of the second embodiment.

### DETAILED DESCRIPTION

The following describes modes (embodiments) for carrying out the present disclosure in detail with reference to the drawings. The present invention is not limited to the description of the embodiments given below. Components described below include those easily conceivable by those skilled in the art or those substantially identical thereto. In addition, the components described below can be combined as appropriate. What is disclosed herein is merely an example, and the present invention naturally encompasses appropriate modifications easily conceivable by those skilled in the art while maintaining the gist of the invention. To further clarify the description, the drawings may schematically illustrate, for example, widths, thicknesses, and shapes of various parts as compared with actual aspects thereof. However, they are merely examples, and interpretation of the present invention is not limited thereto. The same component as that described with reference to an already mentioned drawing is denoted by the same reference numeral through the present specification and the drawings, and detailed description thereof may not be repeated where appropriate.

In this disclosure, when an element is described as being “on” another element, the element can be directly on the other element, or there can be one or more elements between the element and the other element.

### First Embodiment

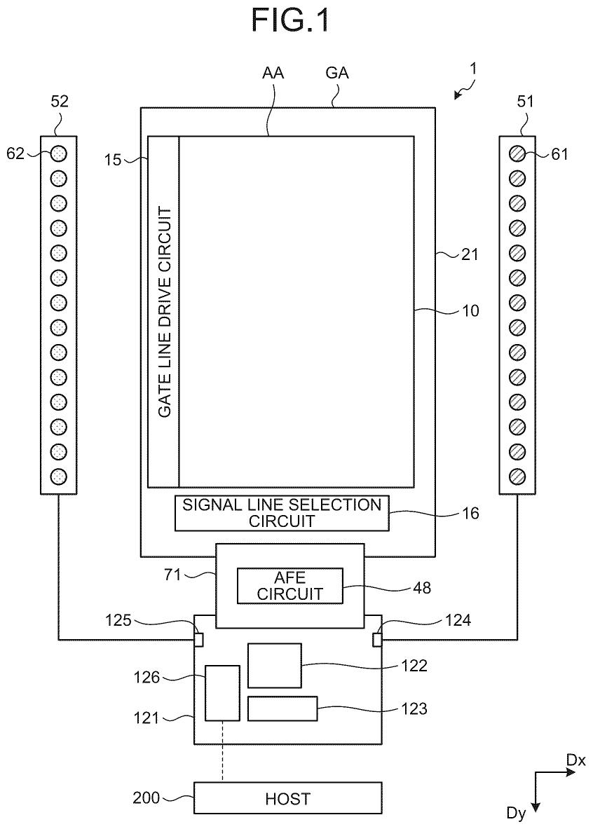

FIG. 1 is a plan view illustrating a detection device according to a first embodiment. As illustrated in FIG. 1, a detection device 1 includes a sensor base member 21, a sensor area 10, a gate line drive circuit 15, a signal line selection circuit 16, an analog front-end (AFE) circuit 48, a control circuit 122, a power supply circuit 123, first light sources 61, and second light sources 62. FIG. 1 illustrates an example in which a first light source base member 51 is provided with a plurality of first light sources 61 and a second light source base member 52 is provided with a plurality of second light sources 62. However, the arrangement of the first and the second light sources 61 and 62 illustrated in FIG. 1 is merely exemplary, and can be modified as appropriate. For example, the first and the second light sources 61 and 62 may be arranged on each of the first and the second base members 51 and 52. In this case, a group including the first light sources 61 and a group including the second light sources 62 may be arranged in a second direction Dy, or the first and the second light sources 61 and 62 may be alternately arranged in the second direction Dy. The first and the second light sources 61 and 62 may be provided on one light source base member, or three or more light source base members.

The detection device 1 is electrically coupled to a host 200. The host 200 is, for example, a higher-level control device for an apparatus (not illustrated) to which the detection device 1 is applied. The host 200 performs a predetermined biometric information acquisition process based on data output from the detection device 1.

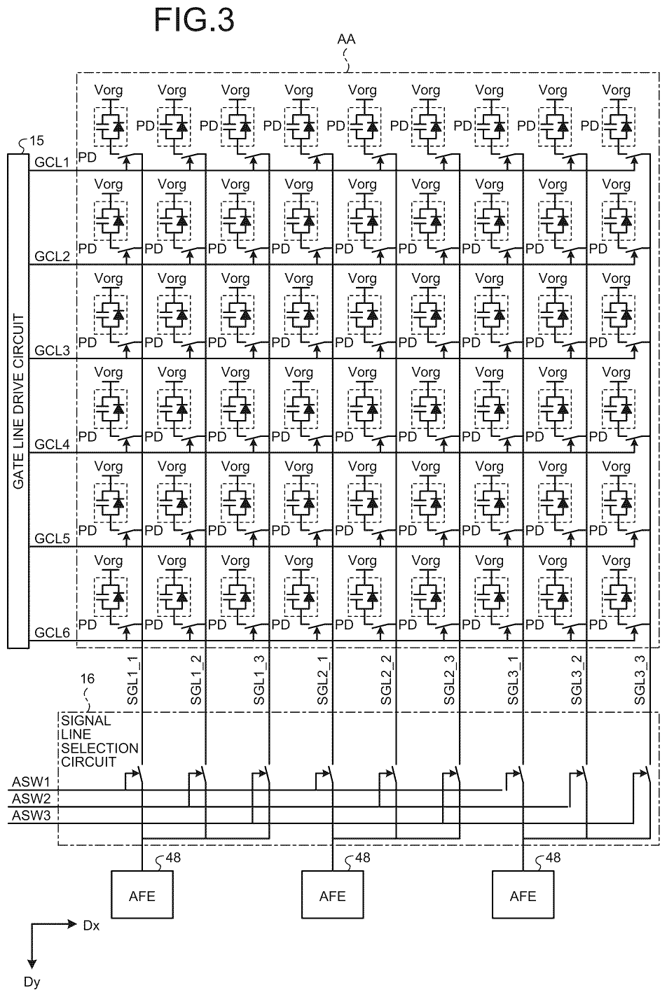

The sensor base member 21 is electrically coupled to a control substrate 121 via a flexible printed circuit board 71. The flexible printed circuit board 71 is provided with the AFE circuit 48. The control substrate 121 is provided with the control circuit 122, the power supply circuit 123, and an output circuit 126. As illustrated in FIG. 3 described later, the AFE circuit 48 is circuitry including a plurality of AFE circuits each of which is provided for a plurality signal lines. In the following descriptions, each of the plurality of AFE circuits included in the AFE circuit 48 as entire circuitry is given the same reference sign “48” and is referred to as the “AFE circuit 48” in some cases.

The control circuit 122 is, for example, a control integrated circuit (IC) that outputs logic control signals. The control circuit 122 may be, for example, a programmable logic device (PLD) such as a field-programmable gate array (FPGA).

The control circuit 122 supplies control signals to the sensor area 10, the gate line drive circuit 15, and the signal line selection circuit 16 to control detection operations in the sensor area 10. The control circuit 122 also supplies control signals to the first and the second light sources 61 and 62 to control lighting and non-lighting of the first and the second light sources 61 and 62.

The power supply circuit 123 supplies voltage signals such as a sensor power supply potential Vorg to the sensor area 10, the gate line drive circuit 15, the signal line selection circuit 16, and the AFE circuit 48. The power supply circuit 123 supplies a power supply voltage to the first and the second light sources 61 and 62.

The output circuit 126 is, for example, a Universal Serial Bus (USB) controller IC, and controls communication between the control circuit 122 and the host 200.

The sensor base member 21 has a detection area AA and a peripheral area GA. The detection area AA is an area where a plurality of optical sensors PD included in the sensor area 10 are provided in a matrix having a row-column configuration. The peripheral area GA is an area between the outer perimeter of the detection area AA and the ends of the sensor base member 21 and is an area not provided with the optical sensors PD.

The gate line drive circuit 15 and the signal line selection circuit 16 are provided in the peripheral area GA. Specifically, the gate line drive circuit 15 is provided in an area extending along the second direction Dy in the peripheral area GA. The signal line selection circuit 16 is provided in an area extending along a first direction Dx in the peripheral area GA and is provided between the sensor area 10 and the AFE circuit 48.

The first direction Dx is one direction in a plane parallel to the sensor base member 21. The second direction Dy is one direction in the plane parallel to the sensor base member 21 and is a direction orthogonal to the first direction Dx. The second direction Dy may non-orthogonally intersect the first direction Dx. A third direction Dz is a direction orthogonal to the first direction Dx and the second direction Dy and is a direction normal to the sensor base member 21.

The first light sources 61 are provided on the first light source base member 51 and arranged along the second direction Dy. The second light sources 62 are provided on the second light source base member 52 and arranged along the second direction Dy. The first light source base member 51 and the second light source base member 52 are electrically coupled to the control circuit 122 and the power supply circuit 123, through terminals 124 and 125, respectively, provided on the control substrate 121.

For example, inorganic light-emitting diodes (LEDs) or organic electroluminescent (EL) diodes (organic light-emitting diodes (OLEDs)) are used as the first and the second light sources 61 and 62. The first and the second light sources 61 and 62 emit first and second light, respectively, having different wavelengths.

The first light emitted from the first light sources 61 is reflected, for example, on a surface of an object to be detected, such as a finger or a wrist of a subject of examination, and is incident on the sensor area 10. As a result, the sensor area 10 can detect a fingerprint by detecting a shape of asperities on the surface of a finger Fg or the like. The second light emitted from the second light sources 62 is, for example, reflected in the finger Fg or the like, or transmitted through the finger Fg or the like, and is incident on the sensor area 10. As a result, the sensor area 10 can detect information on a living body in the finger, the wrist, or the like of the subject of examination. Examples of the information on the living body include, but are not limited to, pulse waves, pulsation, and a vascular image of the subject of examination. That is, the detection device 1 may be configured as a fingerprint detection device that detects a fingerprint or a vein detection device that detects a vascular pattern of, for example, veins.

The first light may have a wavelength of 420 nm to 600 nm, for example, approximately 500 nm, and the second light may have a wavelength of 780 nm to 950 nm, for example, approximately 850 nm. In this case, the first light is blue or green visible light (blue light or green light), and the second light is infrared light. The sensor area 10 can detect a fingerprint based on the first light emitted from the first light sources 61. The second light emitted from the second light sources 62 is reflected in, or transmitted through or absorbed by the object to be detected, and is incident on the sensor area 10. As a result, the sensor area 10 can detect biometric data such as the pulse waves and the vascular image (vascular pattern) as the information on the living body in the finger, the wrist, or the like of the subject of examination.

Alternatively, the first light may have a wavelength of 600 nm to 700 nm, for example, approximately 660 nm, and the second light may have a wavelength of 780 nm to 950 nm, for example, approximately 850 nm. In this case, the sensor area 10 can detect a blood oxygen level in addition to the pulse waves, the pulsation, and the vascular image as the information on the living body based on the first light emitted from the first light sources 61 and the second light emitted from the second light sources 62. In this way, the detection device 1 includes the first and the second light sources 61 and 62, and performs the detection based on the first light and the detection based on the second light, and thereby can detect the various types of information on the living body.

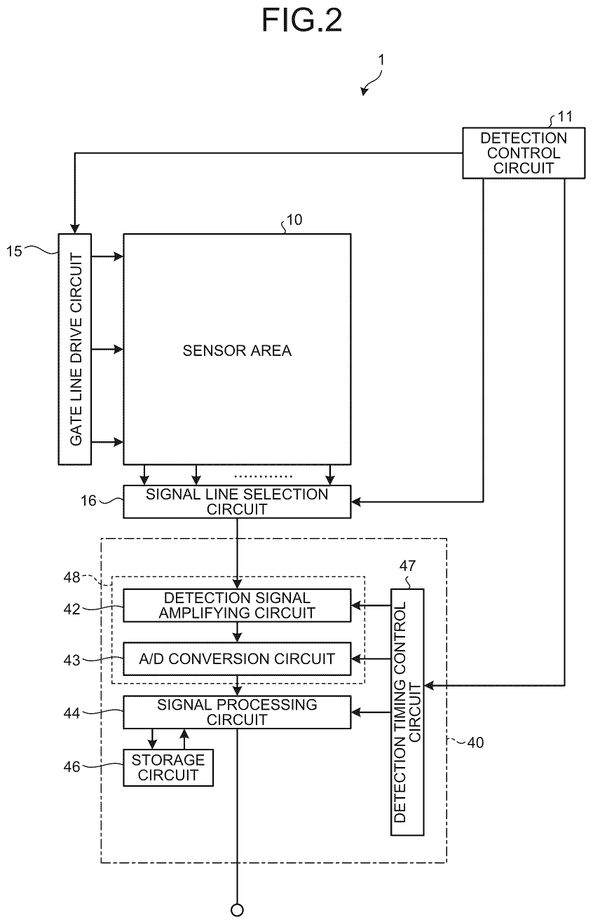

FIG. 2 is a block diagram illustrating a configuration example of the detection device according to the first embodiment. As illustrated in FIG. 2, the detection device 1 further includes a detection control circuit 11 and a detection circuit 40.

The sensor area 10 includes the optical sensors PD. Each of the optical sensors PD included in the sensor area 10 is, for example, an organic photodiode (OPD), and outputs an electrical signal corresponding to light emitted thereto to the signal line selection circuit 16. The sensor area 10 performs detection in response to a gate drive signal supplied from the gate line drive circuit 15. The optical sensor PD may be a silicon photodiode (SiPD), for example.

The detection control circuit 11 is a circuit that supplies respective control signals to the gate line drive circuit 15, the signal line selection circuit 16, and the detection circuit 40 to control operations of these circuits. The detection control circuit 11 also supplies various control signals to the first and the second light sources 61 and 62 to control the lighting and the non-lighting of each group of the first and the second light sources 61 and 62.

The gate line drive circuit 15 is a circuit that drives a plurality of gate lines GCL (refer to FIG. 3) based on various control signals. The gate line drive circuit 15 sequentially or simultaneously selects the gate lines GCL and supplies the gate drive signal to the selected gate lines GCL. Through this operation, the gate line drive circuit 15 selects the optical sensors PD coupled to the gate lines GCL.

The signal line selection circuit 16 is a switch circuit that sequentially or simultaneously selects a plurality of signal lines SGL (refer to FIG. 3). The signal line selection circuit 16 is a multiplexer, for example. The signal line selection circuit 16 electrically couples the selected signal lines SGL to the AFE circuit 48 based on selection signals ASW supplied from the detection control circuit 11. Through this operation, the signal line selection circuit 16 outputs a detection signal of each of the optical sensors PD to the detection circuit 40.

The detection circuit 40 includes the AFE circuit 48, a signal processing circuit 44, a storage circuit 46, and a detection timing control circuit 47. The detection timing control circuit 47 controls the AFE circuit 48 and the signal processing circuit 44 such that they operate in synchronization with each other, based on the control signal supplied from the detection control circuit 11.

The AFE circuit 48 generates a detection value of each of the optical sensors PD based on the detection signal of the optical sensor PD output from the sensor area 10. The AFE circuit 48 is an analog front-end IC, for example.

The AFE circuit 48 includes at least a detection signal amplifying circuit 42 and an analog-to-digital (A/D) conversion circuit 43 (output circuit). The detection signal amplifying circuit 42 converts a variation of a current supplied from the optical sensor PD into a variation of a voltage, and amplifies the conversion result. The A/D conversion circuit 43 samples the voltage signal output from the detection signal amplifying circuit 42 and converts it into a digital detection value.

In the present disclosure, the control circuit 122 includes the signal processing circuit 44 and the storage circuit 46.

The signal processing circuit 44 acquires the biometric data for generating the information on the living body based on the detection values of the optical sensors PD output from the AFE circuit 48. In the present disclosure, the information on the living body includes the pulse waves acquired using the infrared light and/or red light, for example.

The storage circuit 46 stores various types of setting information required when the signal processing circuit 44 acquires the biometric data. The storage circuit 46 may have a configuration including, for example, a random-access memory (RAN), a read-only memory (ROM), and an electrically erasable programmable read-only memory (EEPROM). The storage circuit 46 may be a register circuit or the like.

The following describes a circuit configuration example of the detection device 1. FIG. 3 is a circuit diagram illustrating the detection device according to the first embodiment. As illustrated in FIG. 3, in the sensor area 10, the optical sensors PD are arranged in a matrix having a row-column configuration in the detection area AA. The gate lines GCL extend in the first direction Dx and are each coupled to the optical sensors PD arranged in the first direction Dx. A plurality of gate lines GCL1, GCL2, . . . , GCL6 are arranged in the second direction Dy and are each coupled to the gate line drive circuit 15. In the following description, the gate lines GCL1, GCL2, . . . , GCL6 will each be simply referred to as the gate line GCL when they need not be distinguished from one another. To facilitate understanding of the description, FIG. 3 illustrates the six gate lines GCL. However, this is merely an example, and M gate lines GCL may be arranged (where M is a natural number).

The signal lines SGL extend in the second direction Dy and are each coupled to the optical sensors PD arranged in the second direction Dy. A plurality of signal lines SGL1_1, SGL1_2, SGL1_3, . . . , SGL3_3 are arranged in the first direction Dx and are each coupled to the signal line selection circuit 16 and a reset circuit 17. In the following description, the signal lines SGL1_1, SGL1_2, SGL1_3, . . . , SGL3_3 will each be simply referred to as the signal line SGL when they need not be distinguished from one another. To facilitate understanding of the description, nine signal lines SGL are illustrated. However, this is merely an example, and N signal lines SGL may be arranged (where N is a natural number).

The gate line drive circuit 15 receives various control signals from the control circuit 122 (refer to FIG. 1). The gate line drive circuit 15 sequentially selects the gate lines GCL1, GCL2, . . . , GCL6 in a time-division manner based on the various control signals. The gate line drive circuit 15 supplies the gate drive signal to the selected gate line GCL. This operation supplies the gate drive signal to switches coupled to the respective optical sensors PD, and the optical sensors PD arranged in the first direction Dx are selected. The switch coupled to the optical sensor PD is a switching element including a thin-film transistor, for example, and is configured as an n-channel metal oxide semiconductor (MOS) thin-film transistor (TFT), for example.

The gate line drive circuit 15 may perform different driving for each of detection modes including the detection of the fingerprint and the detection of a plurality of different items of information on a living body (including, for example, the pulse waves, the pulsation, the vascular image, and the blood oxygen level, which are hereinafter called also simply “biometric information”). For example, the gate line drive circuit 15 may collectively drive more than one of the gate lines GCL.

In the signal line selection circuit 16, a plurality of switches are provided correspondingly to the signal lines SGL1_1, SGL1_2, and SGL1_3, . . . , SGL3_3. The switches provided correspondingly to the signal lines SGL are each a switching element including a thin-film transistor, for example, and are each configured as an n-channel metal oxide semiconductor (MOS) thin-film transistor (TFT), for example.

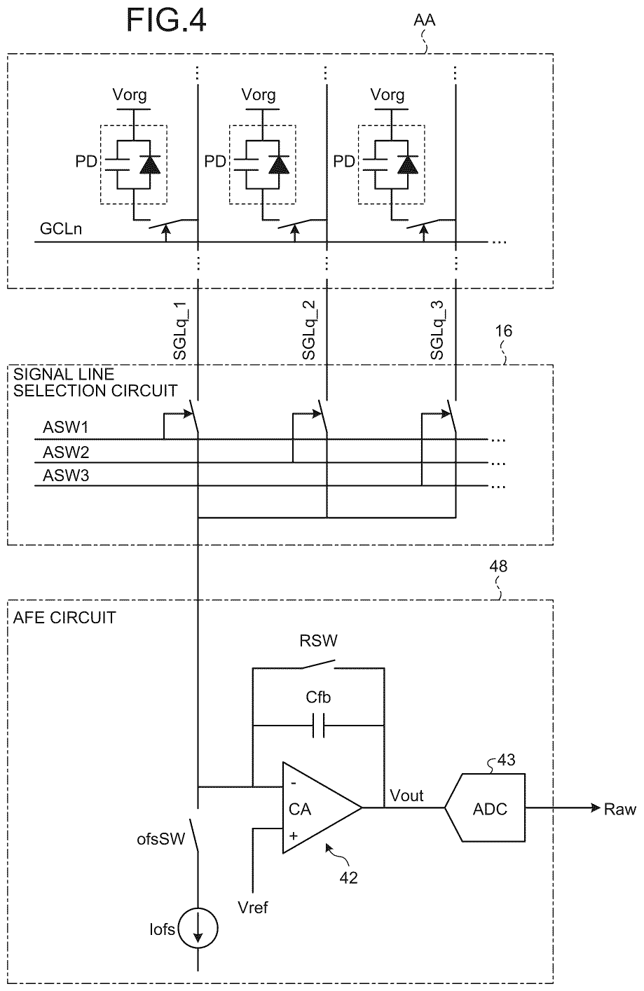

The control circuit 122 (refer to FIG. 1) supplies selection signals ASW1, ASW2, and ASW3 to the signal line selection circuit 16. This operation causes the signal line selection circuit 16 to sequentially select a plurality of signal lines SGL in a time-division manner or simultaneously select the signal lines SGL. Specifically, in the configuration illustrated in FIG. 3, the signal line selection circuit 16 simultaneously selects the signal lines SGL1_1, SGL2_1, and SGL3_1 and couples them to the AFE circuit 48. The signal line selection circuit 16 also simultaneously selects the signal lines SGL1_2, SGL2_2, and SGL3_2 and couples them to the AFE circuit 48. The signal line selection circuit 16 also simultaneously selects the signal lines SGL1_3, SGL2_3, and SGL3_3 and couples them to the AFE circuit 48. Such a configuration can reduce the number of ICs including the AFE circuit 48 or the number of terminals of the ICs in the detection device 1. To facilitate understanding of the description, FIG. 3 illustrates an example in which three of the signal lines SGL are selected in a time-division manner, but this is only an example, and the number of the signal lines SGL selected in a time-division manner may be P (P is a natural number obtained by equally dividing the number M by a number of equal divisions; for example, when the number of equal divisions of the number M of the signal lines is Q, P=M/Q) FIG. 4 is a circuit diagram illustrating a configuration example of the AFE circuit according to the first embodiment. FIG. 4 also illustrates the optical sensors PD. The optical sensors PD are selected by the gate drive signal supplied from the gate line drive circuit 15 to a gate line GCLn (where n is a natural number from 1 to N (N=6 in the example illustrated in FIG. 3)) and are coupled by the signal line selection circuit 16 to the AFE circuit 48 via signal lines SGLq_1, SGLq_2, and SGLq_3 (where q is a natural number from 1 to Q (Q=3 in the example illustrated in FIG. 3)).

The sensor power supply potential Vorg is applied to the cathode of each of the optical sensors PD from the power supply circuit 123. The anode of each of the optical sensors PD is coupled to the AFE circuit 48 via the signal line selection circuit 16 in a time-division manner.

When the optical sensor PD is irradiated with light, a current corresponding to the intensity of the light received by the optical sensor PD flows through the optical sensor PD, and an electric charge is stored in the capacitive element of the optical sensor PD. When the optical sensor PD is selected by the gate line drive circuit 15 and the signal line selection circuit 16, a current corresponding to the electric charge stored in the capacitive element of the optical sensor PD flows to the AFE circuit 48 via the signal line SGL.

The detection signal amplifying circuit 42 of the AFE circuit 48 converts the variation of the current supplied from the optical sensor PD via the signal line SGL into the variation of the voltage, and amplifies the result. The detection signal amplifying circuit 42 includes a differential amplifying circuit CA as a main component.

A reference potential Vref having a fixed potential is supplied to a non-inverting input terminal (+) of the differential amplifying circuit CA, and the signal lines SGL are coupled to an inverting input terminal (−) of the differential amplifying circuit CA via the signal line selection circuit 16. The reference potential Vref is, for example, an approximately half value of a power supply voltage Vadc of the A/D conversion circuit 43 (Vref Vadc/2). The power supply voltage Vadc of the A/D conversion circuit 43 is given as a potential difference between a high potential voltage Vadh and a low potential voltage Vadl supplied to the A/D conversion circuit 43. The low potential voltage Vadl is a ground (GND) potential, for example.

A negative feedback capacitor Cfb and a reset switch RSW are coupled between the inverting input terminal (−) and the output terminal of the differential amplifying circuit CA. In the present embodiment, a constant current source is coupled to the inverting input terminal (−) via an offset switch ofsSW.

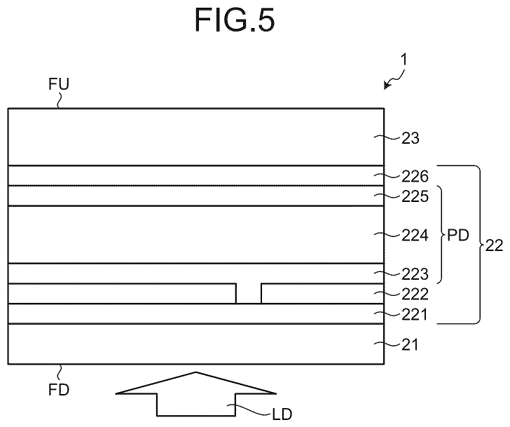

The following describes a configuration of the optical sensor PD. FIG. 5 is a schematic partial sectional view of the optical sensor according to the first embodiment. The sensor area 10 of the detection device 1 includes the sensor base member 21, a sensor structure 22, and a protective film 23. The sensor base member 21 is, for example, an insulating base member formed of a film-like resin.

The sensor structure 22 includes a TFT layer 221, an anode electrode (lower electrode) 222, the optical sensor PD, and a cathode electrode (upper electrode) 226.

The TFT layer 221 is provided with various types of wiring such as the gate lines GCL and the signal lines SGL. The sensor base member 21 and the TFT layer 221 are a drive circuit that drives the sensor and are also called a backplane.

The optical sensor PD includes an active layer 224, an electron transport layer (lower buffer layer) 223 provided between the active layer 224 and the anode electrode (lower electrode) 222, and a hole transport layer (upper buffer layer) 225 provided between the active layer 224 and the cathode electrode (upper electrode) 226. In other words, the electron transport layer (lower buffer layer) 223, the active layer 224, and the hole transport layer (upper buffer layer) 225 of the optical sensor PD are stacked in this order in a direction orthogonal to the sensor base member 21.

The active layer 224 changes in characteristics (for example, voltage-current characteristics and resistance value) according to light emitted thereto. An organic material is used as a material of the active layer 224. Specifically, the active layer 224 has a bulk heterostructure containing a mixture of a p-type organic semiconductor and an n-type fullerene derivative ((6,6)-phenyl-C₆₁-butyric acid methyl ester (PCBM)) that is an n-type organic semiconductor. As the active layer 224, low-molecular-weight organic materials can be used including, for example, fullerene (C₆₀), phenyl-C₆₁-butyric acid methyl ester (PCBM), copper phthalocyanine (CuPc), fluorinated copper phthalocyanine (F₁₆CuPc), 5,6,11,12-tetraphenyltetracene (rubrene), and perylene diimide (PDI) (derivative of perylene).

The active layer 224 can be formed by a vapor deposition process (dry process) using any of the low-molecular-weight organic materials listed above. In this case, the active layer 224 may be, for example, a multilayered film of CuPc and F₁₆CuPc, or a multilayered film of rubrene and C₆₀. The active layer 224 can also be formed by a coating process (wet process). In this case, the active layer 224 is made using a material obtained by combining any of the above-listed low-molecular-weight organic materials with a high-molecular-weight organic material. As the high-molecular-weight organic material, for example, poly(3-hexylthiophene) (P3HT) and F8-alt-benzothiadiazole (F8BT) can be used. The active layer 224 can be a film made of a mixture of P3HT and PCBM, or a film made of a mixture of F8BT and PDI.

The electron transport layer (lower buffer layer) 223 and the hole transport layer (upper buffer layer) 225 are provided to facilitate electrons and holes generated in the active layer 224 to reach the anode electrode (lower electrode) 222 or the cathode electrode (upper electrode) 226. The electron transport layer (lower buffer layer) 223 is in direct contact with the top of the anode electrode (lower electrode) 222. The active layer 224 is in direct contact with the top of the electron transport layer (lower buffer layer) 223. Polyethylenimine ethoxylated (PEIE) is used as a material of the electron transport layer (lower buffer layer) 223.

The hole transport layer (upper buffer layer) 225 is in direct contact with the top of the active layer 224, and the cathode electrode (upper electrode) 226 is in direct contact with the top of the hole transport layer (upper buffer layer) 225. The hole transport layer (upper buffer layer) 225 is a metal oxide layer. For example, tungsten oxide (WO₃) or molybdenum oxide is used as the metal oxide layer.

The materials and the manufacturing methods of the electron transport layer (lower buffer layer) 223, the active layer 224, and the hole transport layer (upper buffer layer) 225 are merely exemplary, and other materials and manufacturing methods may be used.

The anode electrode (lower electrode) 222 faces the cathode electrode (upper electrode) 226 with the optical sensor PD interposed therebetween. A light-transmitting conductive material such as indium tin oxide (ITO) is used as the cathode electrode (upper electrode) 226. A metal material such as silver (Ag) or aluminum (Al) is used as the anode electrode (lower electrode) 222. Alternatively, the anode electrode (lower electrode) 222 may be an alloy material containing at least one or more of these metal materials.

The anode electrode (lower electrode) 222 can be formed as a light-transmitting transflective electrode by controlling the film thickness of the anode electrode (lower electrode) 222. For example, the anode electrode (lower electrode) 222 is formed of an Ag thin film having a thickness of 10 nm so as to have light transmittance of approximately 60%. In this case, the optical sensor PD can detect, for example, first light LD entering from a first surface FD side.

The protective film 23 is provided on a second surface FU so as to cover the cathode electrode (upper electrode) 226. The protective film 23 is a passivation film and is provided to protect the optical sensor PD.

In the present disclosure, as a specific example of the information on the living body acquired by the detection device 1, the following describes an example of acquiring the pulse waves serving as the biometric information for calculating the oxygen saturation level in blood (hereinafter, called the blood oxygen saturation level (SpO₂)).

In this example, for example, red visible light (red light) having a wavelength of 600 nm to 700 nm, specifically approximately 660 nm is employed as the first light emitted from the first light sources 61, and infrared light having a wavelength of 780 nm to 950 nm, specifically approximately 850 nm is employed as the second light emitted from the second light sources 62. In the case of acquiring the human blood oxygen saturation level (SpO₂), a first pulse wave acquired using the first light (red light) and a second pulse wave acquired using the second light (infrared light) are used.

The blood oxygen saturation level (SpO₂) can be obtained by: denoting the alternating-current (AC) component of the first pulse wave as AC(Red), the direct-current (DC) component of the first pulse wave as DC(Red), the AC component of the second pulse wave as AC(IR), and the DC component of the second pulse wave as DC(IR); calculating the value of R given by Expression (1) below; and applying the value of R to a calibration curve given by Expression (2) below (where a and b are calibration coefficients).

$$R = \{ AC ( Red ) / DC ( Red ) \} / \{ AC ( IR ) / DC ( I R ) \} \qquad ( 1 )$$

$${Sp O}_{2} = b - a \times R \qquad ( 2 )$$

Since the amount of light absorbed changes depending on the amount of oxygen taken in by hemoglobin, the optical sensor PD detects the amount of light obtained by subtracting the light absorbed by blood (hemoglobin) from the applied first and second light. Most of the oxygen in blood is reversibly bound to hemoglobin in red blood cells, and a small portion thereof is dissolved in blood plasma. More specifically, the value indicating what percentage of a permissible amount of oxygen is bound to blood as a whole is called the oxygen saturation level (SpO₂). At the two wavelengths of the first light and the second light, the blood oxygen saturation level can be calculated from the amount obtained by subtracting the light absorbed by blood (hemoglobin) from the applied light.

The blood oxygen saturation level (SpO₂) is determined by the ratio of hemoglobin in blood bound to oxygen (oxygenated hemoglobin (O₂Hb)) to hemoglobin in blood not bound to oxygen (reduced hemoglobin (HHb)). The light absorption characteristics of the red light are represented as HHb>>O₂Hb, indicating that HHb has significantly higher absorbance, while the light absorption characteristics of the infrared light are represented as HHb≈O₂Hb, indicating that O₂Hb has slightly higher absorbance.

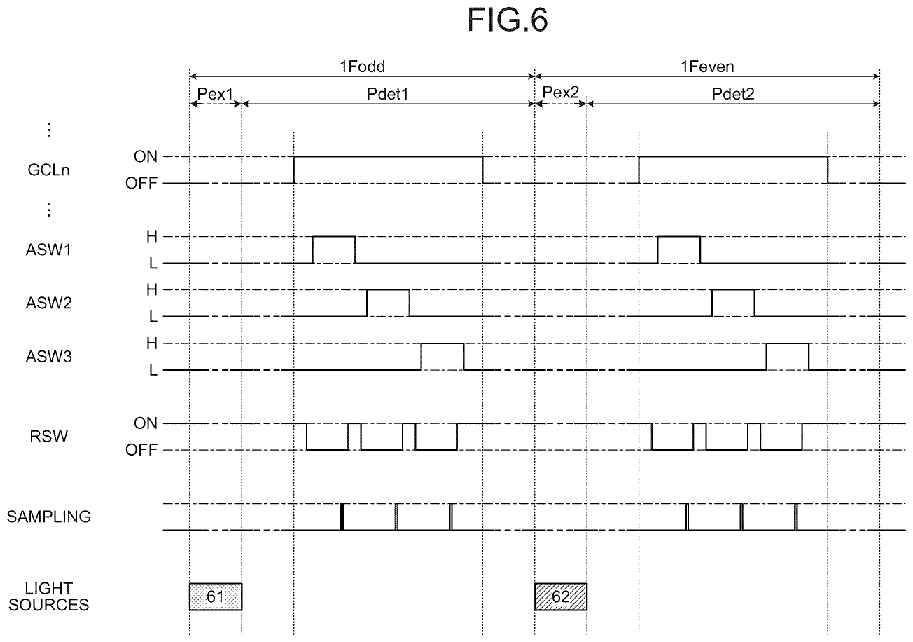

The following describes an operation example of the detection device 1. Herein, first, an operation example according to a comparative example for the first embodiment will be described. FIG. 6 is a timing waveform diagram illustrating the operation example of a detection device according to the comparative example for the first embodiment. In the operation example of the detection device according to the comparative example for the first embodiment illustrated in FIG. 6, no constant current source is coupled to the inverting input terminal (−) of the differential amplifying circuit CA.

In the example illustrated in FIG. 6, exposure periods Pex1 and Pex2 and readout periods Pdet1 and Pdet2 are provided in an odd-numbered frame 1Fodd and an even-numbered frame 1Feven, respectively. In the configuration illustrated in FIG. 3, the gate line drive circuit 15 sequentially scans from the gate line GCL1 to the gate line GCL6 in each of the readout periods Pdet1 and Pdet2.

As illustrated in FIG. 6, the control circuit 122 (detection control circuit 11) causes the first light sources 61 to be on and causes the second light sources 62 to be off, during the exposure period Pex1 of the odd-numbered frame 1Fodd. The control circuit 122 causes the first light sources 61 to be off and causes the second light sources 62 to be on, during the exposure period Pex2 of the even-numbered frame 1Feven.

Thus, the first and the second light sources 61 and 62 are controlled to be turned on or off in a time-divisional manner for each frame. As a result, the detection value of the first light detected by the optical sensor PD and the detection value of the second light detected by the optical sensor PD are output to the AFE circuit 48 in a time-division manner. Hereinafter, when the odd-numbered frame 1Fodd and the even-numbered frame 1Feven need not be distinguished from each other, the odd-numbered frame 1Fodd and the even-numbered frame 1Feven are each simply referred to as “each frame 1F”; the exposure periods Pex1 and Pex2 are each referred to as an “exposure period Pex”; and the readout periods Pdet1 and Pdet2 are each simply referred to as a “read period Pdet”.

During the exposure period Pex, a current Iphoto corresponding to the intensity of the light applied to the optical sensor PD flows through the optical sensor PD, and an electric charge is stored in the capacitive element of the optical sensor PD. At this time, an electric charge Qphoto stored in the capacitive element of the optical sensor PD is a value obtained by multiplying the current Iphoto flowing through the optical sensor PD by the time length of the exposure period Pex (Qphoto=Iphoto×Pex).

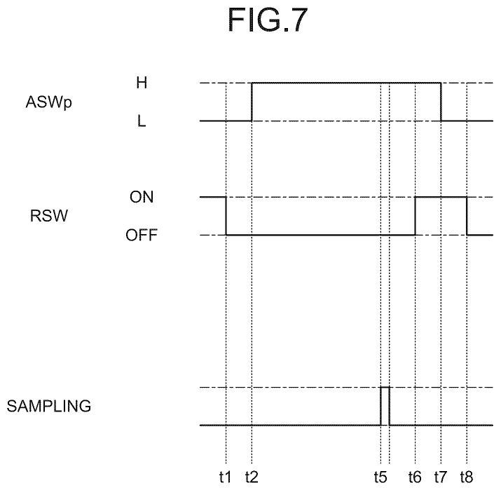

During a selection period of the gate line GCLn, the selection signals ASW1, ASW2, and ASW3 are sequentially controlled to be high (hereinafter, also referred to as “H-controlled”), and the signal lines SGL are sequentially coupled to the AFE circuit 48. High periods (hereinafter, also referred to as “H periods”) during which the selection signals ASW1, ASW2, and ASW3 are respectively high, are each a readout period for each of the optical sensors PD. FIG. 7 is an enlarged diagram of the readout period for each of the optical sensors.

In a period including the exposure period Pex before time t1, the reset switch RSW is controlled to be on and a reset state is established. At time t1 of the readout period for each of the optical sensors illustrated in FIG. 7, the reset switch RSW is controlled to be off to terminate the reset state, and then at time t2, a selection signal ASWp (where p is a natural number 1 to P (P=3 in the example illustrated in FIG. 3)) is controlled to be high (H-controlled). As a result, a current corresponding to the electric charge stored in the capacitive element of the optical sensor PD flows to the AFE circuit 48 via the signal line SGL during the exposure period Pex, and an electric charge is stored in the negative feedback capacitor Cfb of the AFE circuit 48. At this time, a voltage signal Vout (hereinafter, also simply referred to as an “output voltage signal Vout”) output from the detection signal amplifying circuit 42 drops from the reference potential Vref to a value corresponding to the electric charge stored in the negative feedback capacitor Cfb of the AFE circuit 48.

The output voltage signal Vout of the detection signal amplifying circuit 42 is taken into the A/D conversion circuit 43 at a sampling time of time t5, and is converted into a digital detection value Raw.

When the reset switch RSW is controlled to be on at the subsequent time t6, the anode potential of the optical sensor PD and the output voltage of the detection signal amplifying circuit 42 are reset to the reference potential Vref, and the electric charge stored in the negative feedback capacitor Cfb of the detection signal amplifying circuit 42 is reset. Then, at time t7, the selection signal ASWp is controlled to be low (L-controlled), and at time t8, the reset switch RSW is controlled to be off.

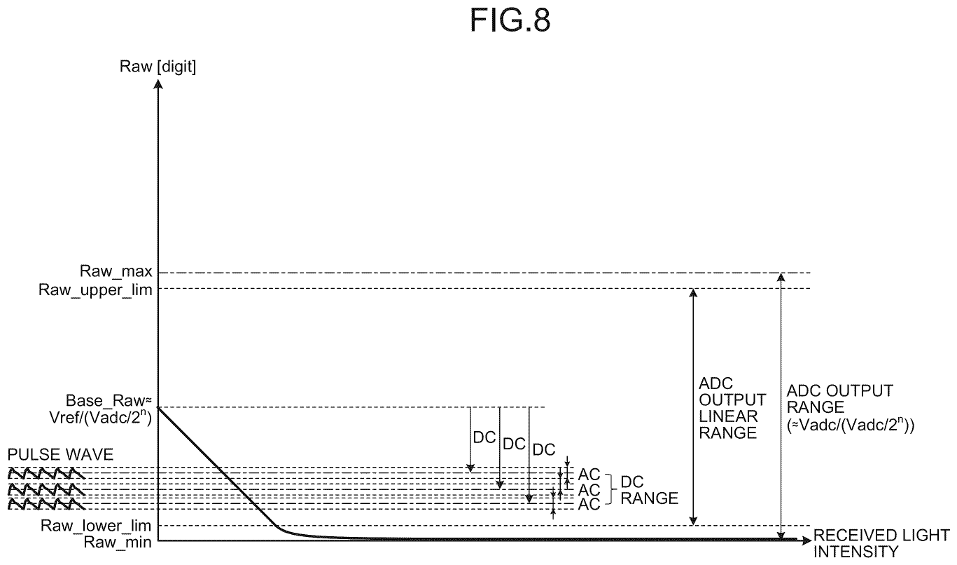

FIG. 8 is a diagram illustrating a correspondence relation between the detection value of the AFE circuit and the received light intensity of the optical sensor in the operation example of the detection device according to the comparative example for the first embodiment. In FIG. 8, the vertical axis indicates the digital detection value Raw (digit), and the horizontal axis indicates the intensity of light received (received light intensity) by the optical sensor PD during the exposure period Pex.

In the configuration according to the first embodiment illustrated in FIGS. 3 and 4, the detection value Raw increases as the received light intensity during the exposure period Pex decreases, as illustrated in FIG. 8. That is, the detection value Raw is a value that decreases with increase in the received light intensity during the exposure period Pex, with the maximum value being the detection value when the optical sensor PD is not exposed to light. In other words, the detection value Raw is a value that decreases with increase in the received light intensity during the exposure period Pex, with the maximum value being a value Base_Raw (hereinafter, also referred to as a “reference value Base_Raw”) obtained by converting the output voltage of the detection signal amplifying circuit 42 at the time of reset into a digital value. In the operation example according to the comparative example illustrated in FIGS. 6 and 7, the reference value Base_Raw is approximately Vref/(Vadc/2ⁿ), assuming that the number of gradations of the A/D conversion circuit 43 is 2ⁿ (at a resolution of n bits) (Base_Raw≈Vref/(Vadc/2ⁿ)). The detection value Raw in the operation example of the detection device according to the comparative example for the first embodiment illustrated in FIGS. 6 and 7 is expressed by Expression (3) below.

$$Raw = Vout / ( Vadc / {2}^{n} ) = ( Vref - Qphoto / Cfb ) / ( Vadc / {2}^{n} ) \qquad ( 3 )$$

When the number of gradations that can be output from the A/D conversion circuit 43 (output circuit) is 2ⁿ, a minimum gradation Raw_min (minimum detection value) is “O”, and a maximum gradation Raw_max (maximum detection value) is “2ⁿ−1”. When the resolution of the A/D conversion circuit 43 is 12 bits (n=12), the minimum gradation Raw_min is “0”, and the maximum gradation Raw_max is “4095”. The reference value Base_Raw is set to “2048” for example.

In the example illustrated in FIG. 8, the output range of the A/D conversion circuit 43 is set to approximately Vadc/(Vadc/2¹). In this case, the range in which the detection value Raw as the output value of the A/D conversion circuit 43 changes linearly with respect to a variation in the output voltage signal Vout of the detection signal amplifying circuit 42, is limited to a range from a lower limit gradation Raw_lower_lim to an upper limit gradation Raw_upper_lim. Therefore, in order to maintain the detection accuracy of the detection value Raw, the detection needs to be performed within the range from the lower limit gradation Raw_lower_lim to the upper limit gradation Raw_upper_lim (Raw_lower_lim≤Raw≤Raw_upper_lim). Hereinafter, the range from the lower limit gradation Raw_lower_lim to the upper limit gradation Raw_upper_lim in which the detection value Raw changes linearly with respect to the variation in the output voltage signal Vout is also referred to as the “detection range” in the detection device 1.

The detection range in the detection device 1 is determined by the input/output characteristics of the A/D conversion circuit 43, the power supply voltage range given by the potential difference between the high potential voltage Vadh (in the present disclosure, the power supply voltage Vadc) and the low potential voltage Vadl (for example, the GND potential) supplied to the A/D conversion circuit 43, and so forth. The input/output characteristics of the detection signal amplifying circuit 42 and the power supply voltage range also affect the detection accuracy of the detection value Raw. In order to maintain the detection accuracy of the detection value Raw, the power supply voltage range of the detection signal amplifying circuit 42 needs to be at least within the power supply voltage range of the A/D conversion circuit 43.

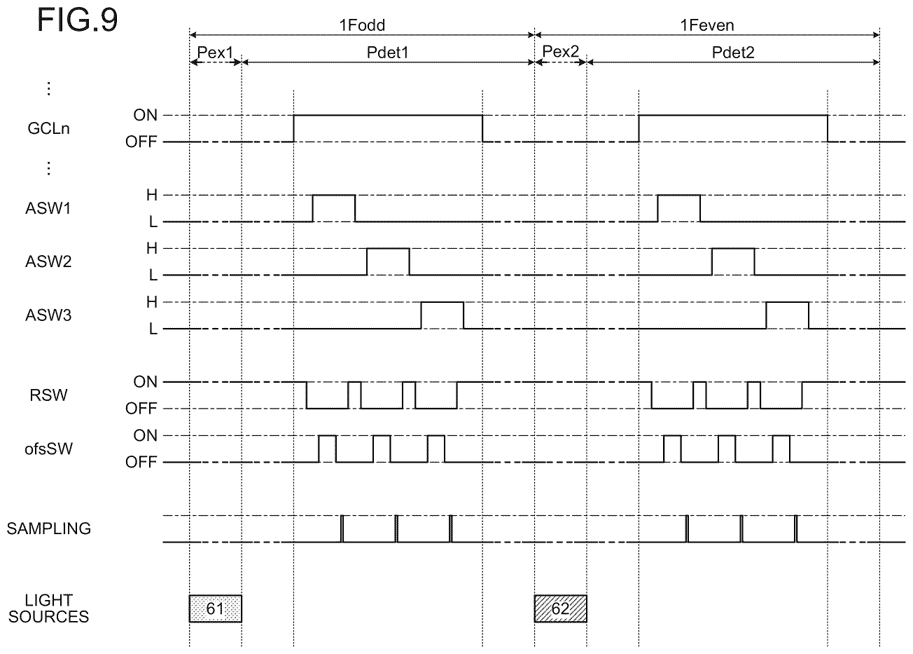

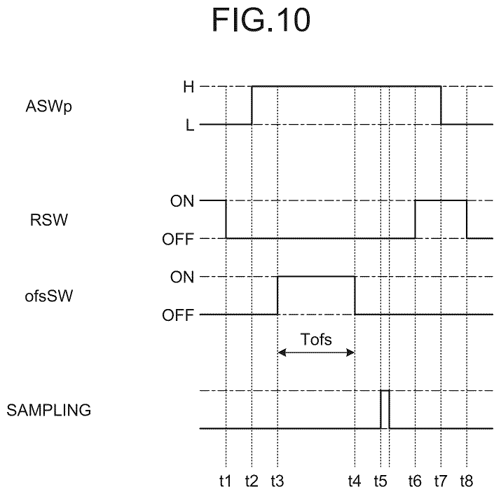

FIG. 9 is a timing waveform diagram illustrating an operation example of the detection device according to the first embodiment. FIG. 10 is an enlarged diagram of the readout period for each of the optical sensors in the timing waveform diagram illustrated in FIG. 9.

Also in the operation example illustrated in FIGS. 9 and 10, the reset switch RSW is controlled to be on and the reset state is established during the period including the exposure period Pex before time t1. In the operation example illustrated in FIGS. 9 and 10, after the reset switch RSW is controlled to be off and the reset state is terminated at time t1 of the readout period for each of the optical sensors illustrated in FIG. 10, the selection signal ASWp is controlled to be high (H-controlled) at time t2. After time t2, the offset switch ofsSW is controlled to be on at time t3, and the offset switch ofsSW is then controlled to be off at time t4 after an offset period Tofs has elapsed. As a result, during the offset period Tofs from time t2 to time t4, a constant current Iofs (hereinafter, also referred to as an “offset current Iofs”) flows out of the optical sensor PD, and the amount of electric charge stored in the negative feedback capacitor Cfb of the detection signal amplifying circuit 42 decreases. An electric charge ΔQofs by which the stored electric charge decreases at this time is Iofs×Tofs (ΔQofs=Iofs×Tofs). As a result, the detection value Raw in the operation example illustrated in FIGS. 9 and 10 is expressed by Expression (4) below.

$$Raw = Vout / ( Vadc / {2}^{n} ) \{ Vref - ( Qphoto - Δ Qofs ) / Cfb \} ( Vadc / {2}^{n} ) \qquad ( 4 )$$

As a result, in the operation example illustrated in FIGS. 9 and 10, the detection value Raw is larger than that in the comparative example illustrated in FIGS. 6 and 7 by a difference value ΔRaw (hereinafter, also referred to as an “offset value ΔRaw”) expressed by Expression (5) below.

$$ΔRaw = ( Δ Qofs / Cfb ) / ( Vadc / {2}^{n} ) = \{ ( Iofs \times Tofs ) / Cfb \} / ( Vadc / {2}^{n} ) \qquad ( 5 )$$

That is, in the operation example of the detection device 1 according to the first embodiment, the offset value ΔRaw can be set by adjusting the offset current Iofs or the offset period Tofs. Specifically, the offset value ΔRaw is set so as to satisfy Expression (6) or (7) below.

$$Raw\_upper \_lim \geq Vref / ( Vadc / {2}^{n} ) + ΔRaw = ( Vref + \{ ( Iofs \times Tofs ) / Cfb \} ) / ( Vadc / {2}^{n} ) \qquad ( 6 )$$

$$Raw\_upper \_lim \times ( V adc / {2}^{n} ) \geq Vref + ΔRaw \times ( Vadc / {2}^{n} ) = Vref + \{ ( Iofs \times Tofs ) / Cfb \} \qquad ( 7 )$$

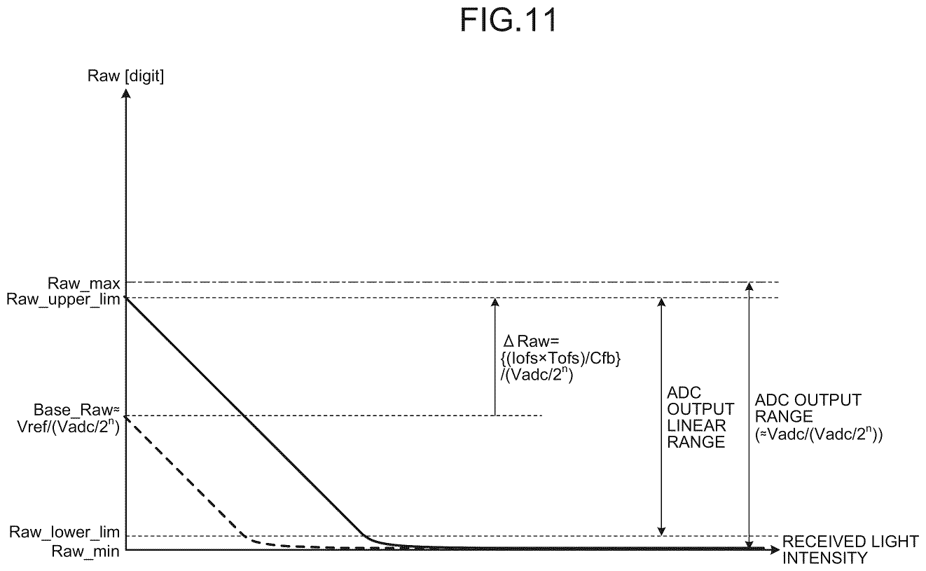

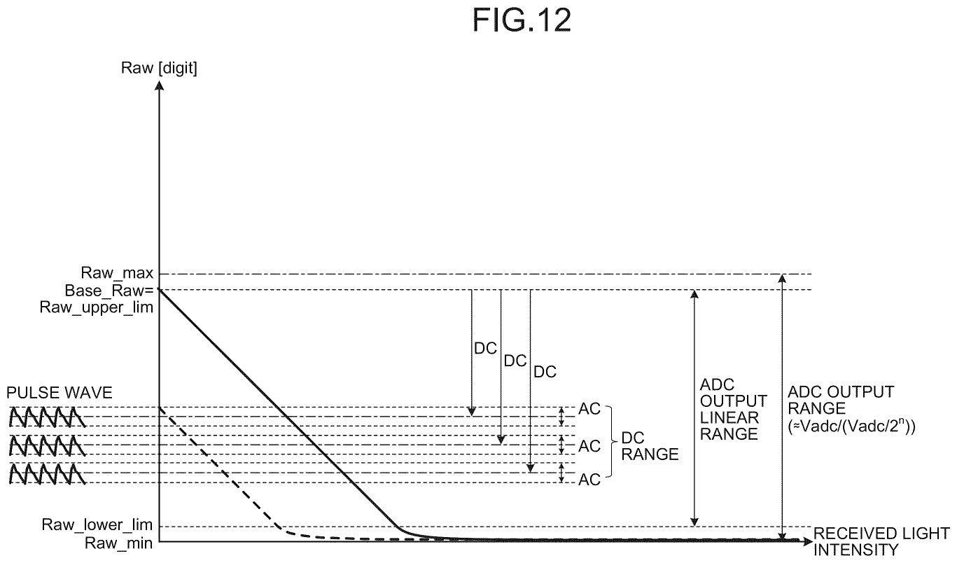

FIG. 11 is a first diagram illustrating the correspondence relation between the detection value of the AFE circuit and the received light intensity of the optical sensor in a first operation example of the detection device according to the first embodiment. FIG. 12 is a second diagram illustrating the correspondence relation between the detection value of the AFE circuit and the received light intensity of the optical sensor in the first operation example of the detection device according to the first embodiment. In the first operation example illustrated in FIGS. 11 and 12, the dashed line illustrates the correspondence relation between the detection value of the AFE circuit and the received light intensity of the optical sensor in the operation example of the detection device according to the comparative example for the first embodiment illustrated in FIG. 8.

FIGS. 11 and 12 illustrate an example in which the offset value ΔRaw is set so that the reference value Base_Raw equals the upper limit gradation Raw_upper_lim (Base_Raw=Raw_upper_lim), as illustrated in FIG. 11. As a result, as illustrated in FIG. 12, by increasing the received light intensity during the exposure period Pex, the AC components (AC(Red) and AC(IR), refer to Expression (1) above) of the pulse waves within the detection range can be made larger than those in the example illustrated in FIG. 8, and the detection accuracy of waveforms of the pulse waves can be improved. To increase the received light intensity during the exposure period Pex, for example, the emission intensity of the light sources (first and second light sources 61 and 62) may be increased or the negative feedback capacitor Cfb may be reduced.

For example, in a configuration of detecting the presence or absence of the object to be detected in the detection area AA, the reference value Base_Raw needs to be within the detection range (Base_Raw≤Raw_upper_lim), as illustrated in FIGS. 11 and 12. In the example of acquiring the pulse waves, even when the reference value Base_Raw is made to match the upper limit gradation Raw_upper_lim (Base_Raw=Raw_upper_lim) as illustrated in FIGS. 11 and 12, the AC components (AC(Red) and AC(IR), refer to Expression (1) above) of the pulse waves are small with respect to the detection range of the A/D conversion circuit 43 (Raw_lower_lim≤Raw≤Raw_upper_lim), and sufficient accuracy may not be obtained.

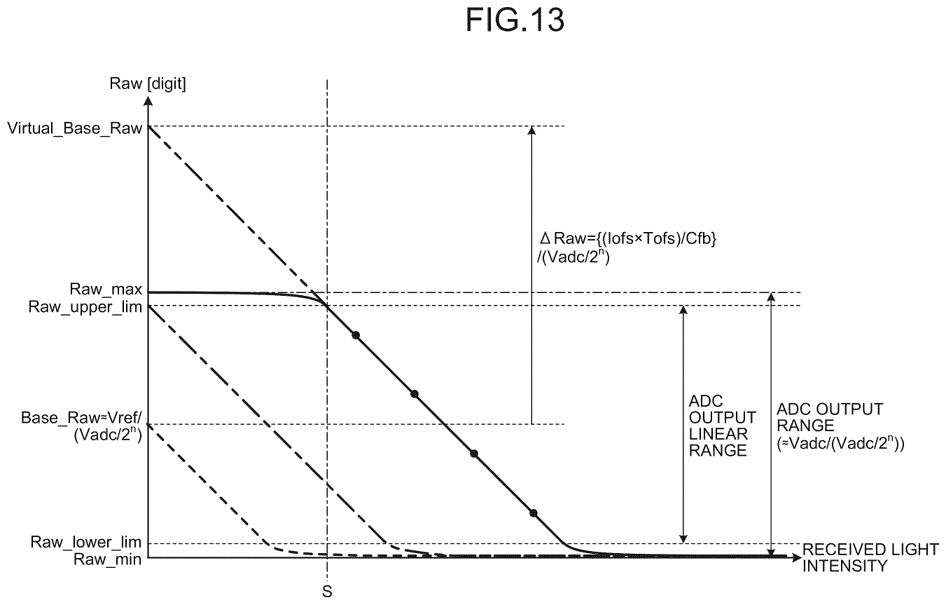

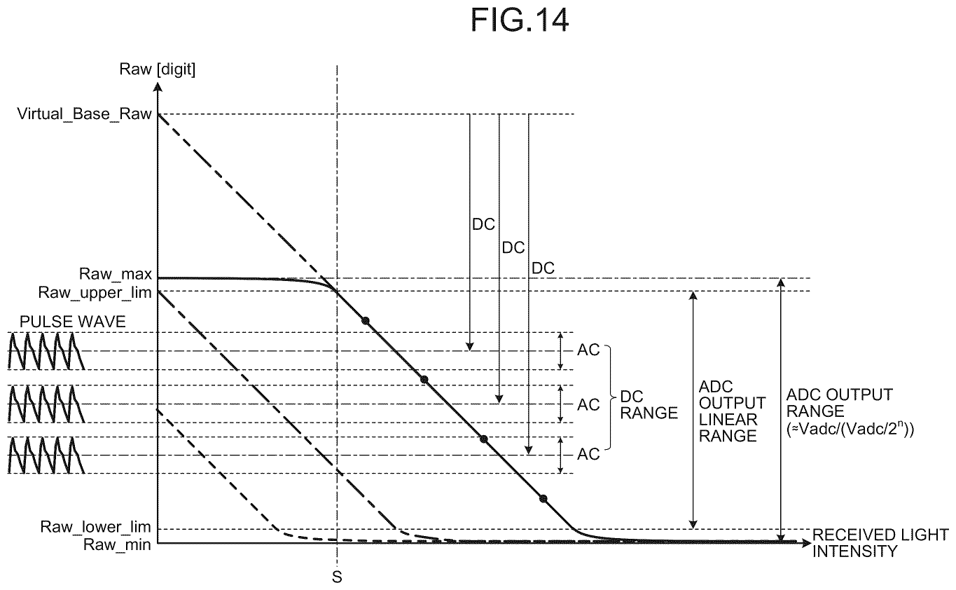

FIG. 13 is a first diagram illustrating the correspondence relation between the detection value of the AFE circuit and the received light intensity of the optical sensor in a second operation example of the detection device according to the first embodiment. FIG. 14 is a second diagram illustrating the correspondence relation between the detection value of the AFE circuit and the received light intensity of the optical sensor in the second operation example of the detection device according to the first embodiment. In the second operation example of the detection device 1 according to the first embodiment illustrated in FIGS. 13 and 14, the dashed line illustrates the correspondence relation between the detection value of the AFE circuit and the received light intensity of the optical sensor in the operation example of the detection device according to the comparative example for the first embodiment illustrated in FIG. 8. The long dashed short dashed line illustrates the correspondence relation between the detection value of the AFE circuit and the received light intensity of the optical sensor in the first operation example of the detection device according to the first embodiment illustrated in FIGS. 11 and 12.

In the second operation example of the detection device 1 according to the first embodiment, as illustrated in FIG. 13, the offset value ΔRaw is larger than that in the first operation example illustrated in FIGS. 11 and 12. Specifically, the offset value ΔRaw is set so as to satisfy Expression (8) or (9) below. As a result, as illustrated in FIG. 14, the upper limit of the range of the detection value Raw used for acquiring the pulse waves can be brought closer to the upper limit gradation Raw_upper_lim, and the range of the detection value Raw used for acquiring the pulse waves can be expanded to the upper limit of the detection range of the detection device 1.

$$Raw\_upper \_lim \leq Vref / ( Vadc / {2}^{n} ) + ΔRaw = ( Vref + \{ ( Iofs \times Tofs ) / Cfb \} ) / ( Vadc / {2}^{n} ) \qquad ( 8 )$$

$$Raw\_upper \_lim \times ( V adc / {2}^{n} ) \leq Vref + ΔRaw \times ( Vadc / {2}^{n} ) = Vref + \{ ( Iofs \times Tofs ) / Cfb \} \qquad ( 9 )$$

As a result, as illustrated in FIG. 14, by making the received light intensity during the exposure period Pex larger than that in the example illustrated in FIGS. 11 and 12, the AC components (AC(Red) and AC(IR), refer to Expression (1) above) of the pulse waves within the detection range can be further increased, and the detection accuracy of the waveforms of the pulse waves can be more improved than in the example illustrated in FIGS. 11 and 12.

In the second operation example of the detection device 1 according to the first embodiment illustrated in FIGS. 13 and 14, in a region where the received light intensity of the optical sensor PD during the exposure period Pex is smaller than a predetermined value S, the detection value Raw is non-linear with respect to the variation of the output voltage signal Vout; and as the received light intensity of the optical sensor PD during the exposure period Pex becomes smaller than the predetermined value S, the detection value Raw is limited to the maximum gradation Raw_max of the A/D conversion circuit 43. In other words, in a region where the output voltage signal Vout of the detection signal amplifying circuit 42 exceeds a predetermined value, the detection value Raw is non-linear with respect to the variation of the output voltage signal Vout; and as the received light intensity of the optical sensor PD during the exposure period Pex becomes smaller than the predetermined value S, the detection value Raw becomes limited to the maximum gradation Raw_max of the A/D conversion circuit 43. That is, in the second operation example of the detection device 1 according to the first embodiment, the detection value Raw is limited to the maximum gradation Raw_max (maximum detection value) of the A/D conversion circuit (output circuit) 43 when the light sources (first and second light sources 61 and 62) are off.

The region where the detection value Raw is non-linear beyond the upper limit gradation Raw_upper_lim is a region outside the detection range in the detection device 1. If the detection range of the detection device 1 is assumed to be infinite, the detection value Raw will be linear over the entire range of the output voltage signal Vout. The long dashed double-short dashed line illustrated in FIGS. 13 and 14 indicates a hypothetical detection value when the detection range of the detection device 1 is assumed to be infinite in the region where the detection value Raw exceeds the upper limit gradation Raw_upper_lim. As described above, the detection value Raw is actually limited to the maximum gradation Raw_max of the A/D conversion circuit 43.

In order to calculate the DC components (DC(Red) and DC(IR), refer to Expression (1) above) of the pulse waves used to calculate the blood oxygen saturation level (SpO₂), a virtual reference value Virtual_Base_Raw needs to be set instead of the reference value Base_Raw. The following describes methods for setting the virtual reference value Virtual_Base_Raw in the second operation example of the detection device 1 according to the first embodiment.

### First Method for Setting Virtual Reference Value According to First Embodiment

As illustrated in FIG. 13, the virtual reference value Virtual_Base_Raw can be calculated by adding the offset value ΔRaw (=(ΔQofs/Cfb)/(Vadc/2ⁿ)) to the reference value Base_Raw in the operation example according to the comparative example for the first embodiment in a state where the optical sensor PD is not exposed to light during the exposure period Pex. In other words, the virtual reference value Virtual_Base_Raw can be calculated by adding the offset value ΔRaw to the reference value Base_Raw when the offset period Tofs is set to approximately zero in the second operation example of the detection device 1 according to the first embodiment in the state where the optical sensor PD is not exposed to light during the exposure period Pex. In this case, the virtual reference value Virtual_Base_Raw is expressed by Expression (10) below.

$$Virtual\_Base \_Raw = Base\_Raw + ΔRaw = ( Vref + \{ ( Iofs \times Tofs ) / Cfb \} ) / ( Vadc / {2}^{n} ) \qquad ( 10 )$$

### Second Method for Setting Virtual Reference Value According to First Embodiment

There is variation between the optical sensors PD. Therefore, the reference value Base_Raw is set to a value obtained for each of the optical sensors PD. The reference value Base_Raw is a value obtained when the offset period Tofs is set to approximately zero in the second operation example of the detection device 1 according to the first embodiment while the optical sensor PD is not exposed to light during the exposure period Pex. The reference value Base_Raw is, for example, a value obtained and set at shipment or the like of the detection device 1. When Base_Raw(m, n) denotes the reference value of the optical sensor PD in the mth column and nth row, a virtual reference value Virtual_Base_Raw(m, n) for the optical sensor PD in the mth column and nth row is expressed by Expression (11) below.

$$Virtual\_Base \_Raw ( m , n ) = Base\_Raw ( m , n ) + Δ Raw = Base\_Raw ( m , n ) + \{ ( Iofs \times Tofs ) / Cfb \} / ( Vadc / {2}^{n} ) \qquad ( 11 )$$

### Third Method for Setting Virtual Reference Value According to First Embodiment

There are variations in the offset current Iofs, the offset period Tofs, and the negative feedback capacitor Cfb of the detection signal amplifying circuit 42. Therefore, it is preferred to acquire the detection values Raw at multiple points (four points in the example illustrated in FIG. 13) within the detection range (Raw_lower_lim≤Raw≤Raw_upper_lim) illustrated in FIG. 13, and set the virtual reference value Virtual_Base_Raw(m, n) for each of the optical sensors PD, for example, by linear approximation (straight-line approximation) using a least-squares method. The virtual reference value Virtual_Base_Raw(m, n) for each of the optical sensors PD is, for example, a value obtained and set at shipment or the like of the detection device 1. The method for calculating the virtual reference value Virtual_Base_Raw(m, n) for each of the optical sensors PD is not limited to the linear approximation.

In the first embodiment, the signal processing circuit 44 calculates the virtual reference value Virtual_Base_Raw using any one of the first method for setting the virtual reference value according to the first embodiment, the second method for setting the virtual reference value according to the first embodiment, and the third method for setting the virtual reference value according to the first embodiment described above. By storing the virtual reference value Virtual_Base_Raw set in this way in the storage circuit 46, the DC components (DC(Red) and DC(IR), refer to Expression (1) above) of the pulse waves used to calculate the blood oxygen saturation level (SpO₂) can be calculated. As a result, the accuracy of calculation of the blood oxygen saturation level (SpO₂) using the first pulse wave acquired using the first light (red light) and the second pulse wave acquired using the second light (infrared light) can be improved.

### Modification

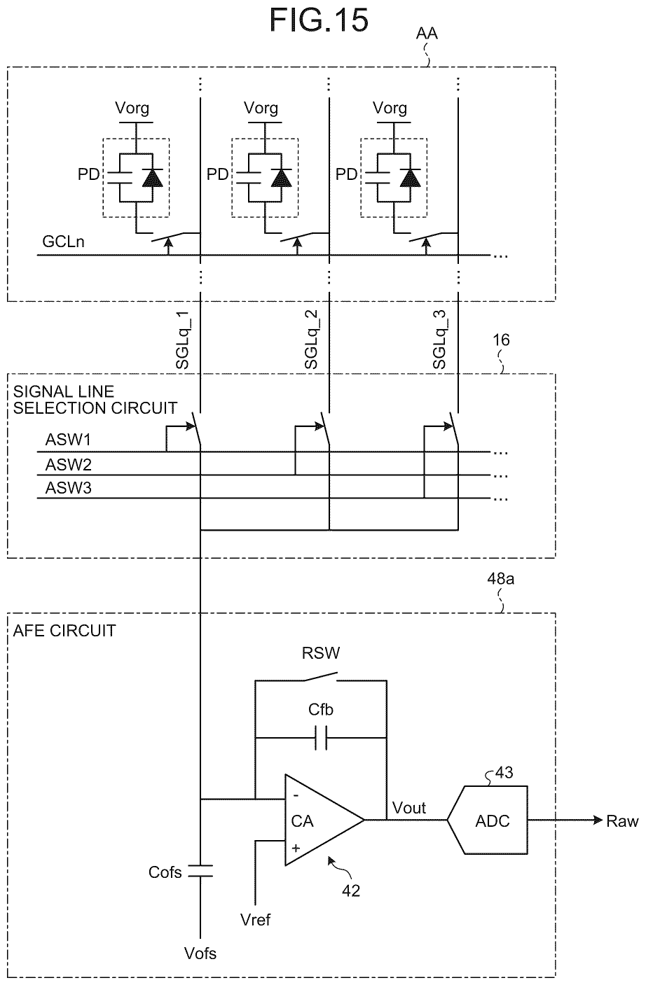

FIG. 15 is a circuit diagram illustrating a configuration example of an AFE circuit according to a modification of the first embodiment. As illustrated in FIG. 15, in the present modification, the inverting input terminal (−) of the differential amplifying circuit CA of the detection signal amplifying circuit 42 included in an AFE circuit 48a receives an offset voltage signal Vofs via an offset capacitor Cofs.

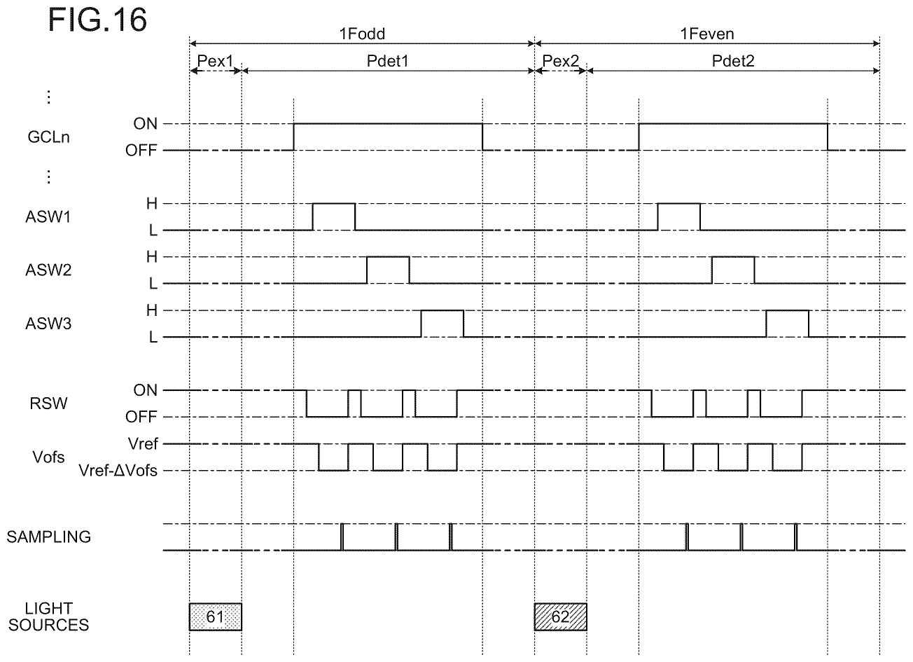

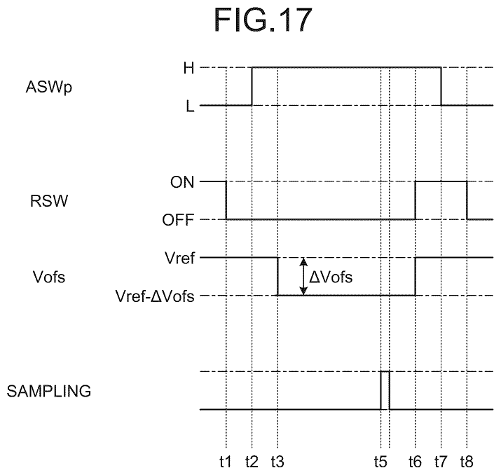

FIG. 16 is a timing waveform diagram illustrating an operation example of the detection device according to the modification of the first embodiment. FIG. 17 is an enlarged diagram of the readout period for each of the optical sensors in the timing waveform diagram illustrated in FIG. 16.

In the operation example of the detection device 1 according to the modification of the first embodiment illustrated in FIGS. 16 and 17, the reference potential Vref is applied as the offset voltage signal Vofs to the offset capacitor Cofs during the period including the exposure period Pex before time t1. As a result, the potential difference between opposite ends of the offset capacitor Cofs is set to approximately zero.

At time t1 of the readout period for each of the optical sensors illustrated in FIG. 17, the reset switch RSW is controlled to be off to terminate the reset state. At time t2 after time t1, the selection signal ASWp is controlled to be high (H-controlled). After time t2, from time t3 to time t6 when the reset switch RSW is controlled to be on, a potential (Vref−ΔVofs) obtained by subtracting an offset potential ΔVofs from the reference potential Vref is applied to the offset capacitor Cofs. As a result, the offset potential ΔVofs is applied between opposite ends of the offset capacitor Cofs, and a part of the electric charge stored in the capacitive element of the optical sensor PD during the exposure period Pex is transferred to the offset capacitor Cofs. The electric charge ΔQofs transferred at this time is ΔVofs×Cofs (ΔQofs=ΔVofs×Cofs).

As a result, in the operation example of the detection device 1 according to the modification of the first embodiment illustrated in FIGS. 16 and 17, the detection value Raw is larger than that in the comparative example illustrated in FIGS. 6 and 7 by the offset value ΔRaw expressed in Expression (12) below.

$$Δ Raw = ( Δ Qofs / Cfb ) / ( Vadc / {2}^{n} ) = \{ ( Δ Vofs \times Cofs ) / Cfb \} / ( Vadc / {2}^{n} ) \qquad ( 12 )$$

That is, in the operation example of the detection device 1 according to the modification of the first embodiment, the offset value ΔRaw can be set by adjusting the offset potential ΔVofs or the offset capacitor Cofs.

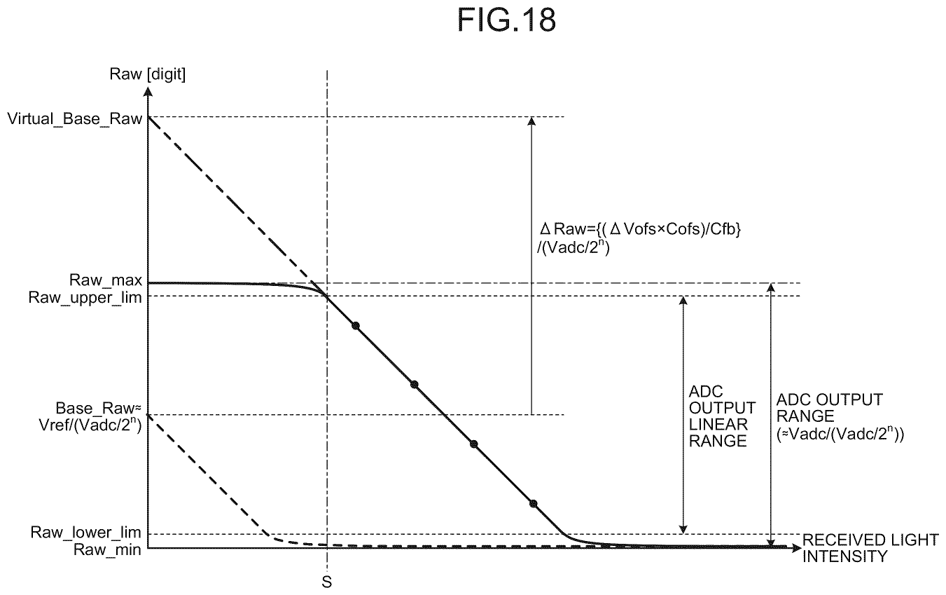

FIG. 18 is a diagram illustrating the correspondence relation between the detection value of the AFE circuit and the received light intensity of the optical sensor in the operation example of the detection device according to the modification of the first embodiment. In FIG. 18, the dashed line illustrates the correspondence relation between the detection value of the AFE circuit and the received light intensity of the optical sensor in the operation example of the detection device according to the comparative example for the first embodiment illustrated in FIG. 8. The long dashed double-short dashed line illustrated in FIG. 18 indicates a hypothetical detection value that is actually limited to the maximum gradation Raw_max of the A/D conversion circuit 43 in the region where the detection value Raw exceeds the upper limit gradation Raw_upper_lim.

In FIG. 18, the offset value ΔRaw is set so as to satisfy Expression (13) or (14) below. With this setting, in the same way as in the second operation example of the detection device 1 according to the first embodiment, the upper limit of the range of the detection value Raw used for acquiring the pulse waves can be brought closer to the upper limit gradation Raw_upper_lim, and the range of the detection value Raw used for acquiring the pulse waves can be expanded to the upper limit of the detection range of the detection device 1.

$$Raw\_upper \_lim \leq Vref / ( Vadc / {2}^{n} ) + Δ Raw = ( Vref + \{ ( Δ Vofs \times Cofs ) / Cfb \} ) / ( Vadc / {2}^{n} ) \qquad ( 13 )$$

$$Raw\_upper \_lim \times ( Vadc / {2}^{n} ) \leq Vref + Δ Raw \times ( Vadc / {2}^{n} ) = Vref + \{ ( Δ Vofs \times Cofs ) / Cfb \} \qquad ( 14 )$$

Thus, in the same way as in the second operation example of the detection device 1 according to the first embodiment, the received light intensity during the exposure period Pex is made larger than that in the first operation example illustrated in FIGS. 11 and 12. To make the received light intensity during the exposure period Pex larger than that in the first operation example, for example, the emission intensity of the light sources (first and second light sources 61 and 62) may be increased or the negative feedback capacitor Cfb may be reduced. As a result, the AC components (AC(Red) and AC(IR), refer to Expression (1) above) of the pulse waves within the detection range can be increased and the detection accuracy of waveforms of the pulse waves can be improved. As described above, the second operation example of the detection device 1 according to the first embodiment is illustrated in FIGS. 13 and 14 and is an operation example with the configuration according to the first embodiment illustrated in FIG. 4.

In the example illustrated in FIG. 18, in the same way as in the second operation example of the detection device 1 according to the first embodiment, in a region where the received light intensity of the optical sensor PD during the exposure period Pex is smaller than the predetermined value S, the detection value Raw is non-linear with respect to the variation of the output voltage signal Vout; and as the received light intensity of the optical sensor PD during the exposure period Pex becomes smaller than the predetermined value S, the detection value Raw is limited to the maximum gradation Raw_max of the A/D conversion circuit 43. In other words, in the same way as in the second operation example of the detection device 1 according to the first embodiment, in a region where the output voltage signal Vout of the detection signal amplifying circuit 42 exceeds a predetermined value, the detection value Raw is non-linear with respect to the variation of the output voltage signal Vout; and as the received light intensity of the optical sensor PD during the exposure period Pex becomes smaller than the predetermined value S, the detection value Raw is limited to the maximum gradation Raw_max of the A/D conversion circuit 43. That is, in the detection device 1 according to the modification of the first embodiment, in the same way as in the second operation example of the detection device 1 according to the first embodiment, the detection value Raw is limited to the maximum gradation Raw_max (maximum detection value) of the A/D conversion circuit (output circuit) 43 when the light sources (first and second light sources 61 and 62) are off.

The region where the detection value Raw is non-linear beyond the upper limit gradation Raw_upper_lim is a region outside the detection range in the detection device 1. If the detection range of the detection device 1 is assumed to be infinite, the detection value Raw will be linear over the entire range of the output voltage signal Vout. The long dashed double-short dashed line illustrated in FIG. 18 indicates a hypothetical detection value when the detection range of the detection device 1 is assumed to be infinite in the region where the detection value Raw exceeds the upper limit gradation Raw_upper_lim. As described above, the detection value Raw is actually limited to the maximum gradation Raw_max of the A/D conversion circuit 43.

In order to calculate the DC components (DC(Red) and DC(IR), refer to Expression (1) above) of the pulse waves used to calculate the blood oxygen saturation level (SpO₂), the virtual reference value Virtual_Base_Raw needs to be set instead of the reference value Base_Raw. The following describes methods for setting the virtual reference value Virtual_Base_Raw in the operation example of the detection device 1 according to the modification of the first embodiment.

### First Method for Setting Virtual Reference Value According to Modification of First Embodiment

As illustrated in FIG. 18, the virtual reference value Virtual_Base_Raw can be calculated by adding the offset value ΔRaw (=(ΔQofs/Cfb)/(Vadc/2ⁿ)) to the reference value Base_Raw in the operation example of the comparative example for the first embodiment in the state where the optical sensor PD is not exposed to light during the exposure period Pex. In other words, the virtual reference value Virtual_Base_Raw can be calculated by adding the offset value ΔRaw to the reference value Base_Raw when the offset potential ΔVofs applied between opposite ends of the offset capacitor Cofs is set to approximately zero in the operation example of the detection device 1 according to the modification of the first embodiment in the state where the optical sensor PD is not exposed to light during the exposure period Pex. In this case, the virtual reference value Virtual_Base_Raw is expressed by Expression (15) below.

$$Virtual\_Base \_Raw = Base\_Raw + Δ Raw = ( Vref + \{ ( Δ Vofs \times Cofs ) / Cfb \} ) / ( Vadc / {2}^{n} ) \qquad ( 15 )$$

### Second Method for Setting Virtual Reference Value According to Modification of First Embodiment

There is variation between the optical sensors PD. Therefore, the reference value Base_Raw is set to a value obtained for each of the optical sensors PD. The reference value Base_Raw is a value obtained when the offset potential ΔVofs applied between opposite ends of the offset capacitor Cofs is set to approximately zero in the operation example of the detection device 1 according to the modification of the first embodiment while the optical sensor PD is not exposed to light during the exposure period Pex. The reference value Base_Raw is, for example, a value obtained and set at shipment or the like of the detection device 1. When Base_Raw(m, n) denotes the reference value of the optical sensor PD in the mth column and nth row, the virtual reference value Virtual_Base_Raw(m, n) for the optical sensor PD in the mth column and nth row is expressed by Expression (16) below.

$$Virtual\_Base \_Raw ( m , n ) = Base\_Raw ( m , n ) + Δ Raw = Base\_Raw ( m , n ) + \{ ( Δ Vofs \times Cofs ) / Cfb \} ) / ( Vadc / {2}^{n} ) \qquad ( 16 )$$

### Third Method for Setting Virtual Reference Value According to Modification of First Embodiment

There are variations in the offset potential ΔVofs, the offset capacitor Cofs, and the negative feedback capacitor Cfb of the detection signal amplifying circuit 42. Therefore, it is preferred to acquire the detection values Raw at multiple points (four points in the example illustrated in FIG. 18) within the detection range (Raw_lower_lim≤Raw≤Raw_upper_lim) illustrated in FIG. 18, and set the virtual reference value Virtual_Base_Raw(m, n) for each of the optical sensors PD, for example, by the linear approximation (straight-line approximation) using the least-squares method. The virtual reference value Virtual_Base_Raw(m, n) for each of the optical sensors PD is, for example, a value obtained and set at shipment or the like of the detection device 1. The method for calculating the virtual reference value Virtual_Base_Raw(m, n) for each of the optical sensors PD is not limited to the linear approximation.

In the modification of the first embodiment, the signal processing circuit 44 calculates the virtual reference value Virtual_Base_Raw using any one of the first method for setting the virtual reference value according to the modification of the first embodiment, the second method for setting the virtual reference value according to the modification of the first embodiment, and the third method for setting the virtual reference value according to the modification of the first embodiment described above. By storing the virtual reference value Virtual_Base_Raw set in this way in the storage circuit 46, the DC components (DC(Red) and DC(IR), refer to Expression (1) above) of the pulse waves used to calculate the blood oxygen saturation level (SpO₂) can be calculated in the same way as in the second operation example of the detection device 1 according to the first embodiment. As a result, the accuracy of calculation of the blood oxygen saturation level (SpO₂) using the first pulse wave acquired using the first light (red light) and the second pulse wave acquired using the second light (infrared light) can be improved.

### Second Embodiment

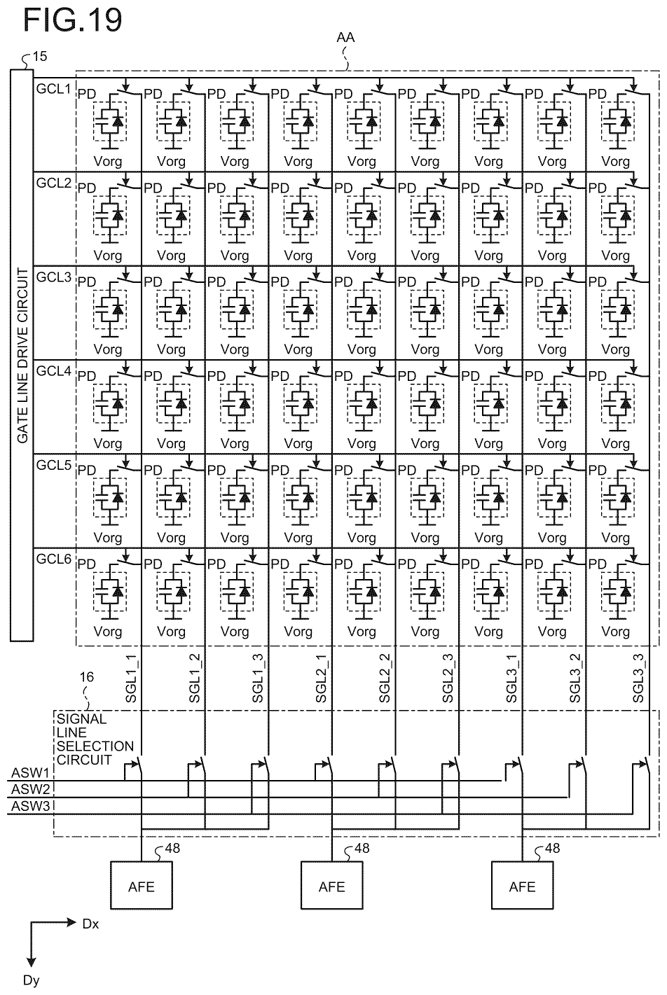

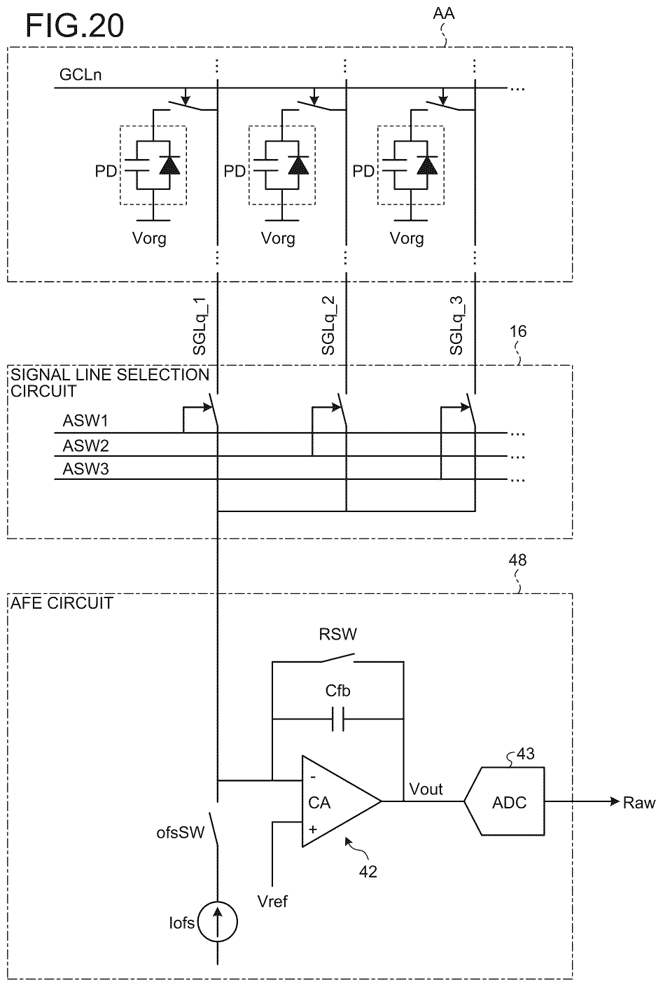

FIG. 19 is a circuit diagram illustrating the detection device according to a second embodiment. FIG. 20 is a circuit diagram illustrating a configuration example of the AFE circuit according to the second embodiment.

In the configuration according to the second embodiment illustrated in FIGS. 19 and 20, the sensor power supply potential Vorg is applied from the power supply circuit 123 to the anode of each of the optical sensors PD. The cathode of each of the optical sensors PD is coupled to the AFE circuit 48 via the signal line selection circuit 16 in a time-division manner.

In the configuration described above, the configuration of the optical sensor PD differs from the configuration of that of the first embodiment illustrated in FIG. 5. Specifically, the anode electrode (lower electrode) 222 illustrated in FIG. 5 corresponds to the cathode electrode (lower electrode) in the configuration according to the second embodiment. The electron transport layer (lower buffer layer) 223 illustrated in FIG. 5 corresponds to the hole transport layer (lower buffer layer) in the configuration according to the second embodiment. The cathode electrode (upper electrode) 226 illustrated in FIG. 5 corresponds to the anode electrode (upper electrode) in the configuration according to the second embodiment.

When the optical sensor PD is irradiated with light, a current corresponding to the intensity of the light received by the optical sensor PD flows through the optical sensor PD, and an electric charge is stored in the capacitive element of the optical sensor PD. When the optical sensor PD is selected by the gate line drive circuit 15 and the signal line selection circuit 16, a current corresponding to the electric charge stored in the capacitive element of the optical sensor PD flows to the AFE circuit 48 via the signal line SGL.

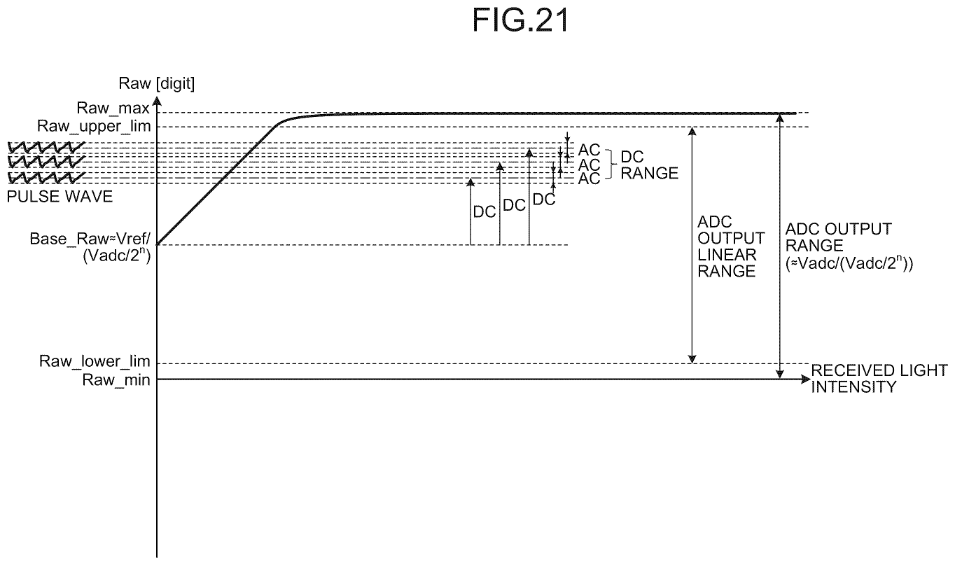

FIG. 21 is a diagram illustrating the correspondence relation between the detection value of the AFE circuit and the received light intensity of the optical sensor in an operation example of a detection device according to a comparative example for the second embodiment. In FIG. 21, the vertical axis indicates the digital detection value Raw (digit), and the horizontal axis indicates the intensity of light received (received light intensity) by the optical sensor PD during the exposure period Pex. The operation example of the detection device according to the comparative example for the second embodiment is the same as that of the detection device according to the comparative example for the first embodiment, and therefore, will not be described in detail.

In the configuration according to the second embodiment illustrated in FIGS. 19 and 20, the detection value Raw decreases as the received light intensity during the exposure period Pex decreases, as illustrated in FIG. 21. That is, the detection value Raw is a value that increases with increase in the received light intensity during the exposure period Pex, with the minimum value being the detection value when the optical sensor PD is not exposed to light. In other words, the detected value Raw is a value that increases with increase in the received light intensity during the exposure period Pex, with the minimum value being the reference value Base_Raw. In the operation example according to the comparative example for the second embodiment, the reference value Base_Raw is approximately Vref/(Vadc/2ⁿ), assuming that the number of gradations of the A/D conversion circuit 43 is 2ⁿ (at a resolution of n bits) (Base_Raw≈Vref/(Vadc/2ⁿ)). The detection value Raw in the operating example of the detection device according to the comparative example for the second embodiment is expressed by Expression (17) below.

$$Raw = Vout / ( Vadc / {2}^{n} ) = ( Vref + Qphoto / Cfb ) / ( Vadc / {2}^{n} ) \qquad ( 17 )$$

When the number of gradations that can be output from the A/D conversion circuit 43 is 2ⁿ, the minimum gradation Raw_min is “0”, and the maximum gradation Raw_max is “2ⁿ−1”. When the resolution of the A/D conversion circuit 43 is 12 bits (n=12), the minimum gradation Raw_min is “0”, and the maximum gradation Raw_max is “4095”.

In the example illustrated in FIG. 21, the output range of the A/D conversion circuit 43 is set to approximately Vadc/(Vadc/2n). In this case, the range in which the detection value Raw as the output value of the A/D conversion circuit 43 linearly changes with the output voltage signal Vout of the detection signal amplifying circuit 42, is limited to the range from the lower limit gradation Raw_lower_lim to the upper limit gradation Raw_upper_lim. Therefore, in order to maintain the detection accuracy of the detection value Raw, the detection needs to be performed within the detection range from the lower limit gradation Raw_lower_lim to the upper limit gradation Raw_upper_lim (Raw_lower_lim≤Raw≤Raw_upper_lim) in the same way as in the first embodiment.

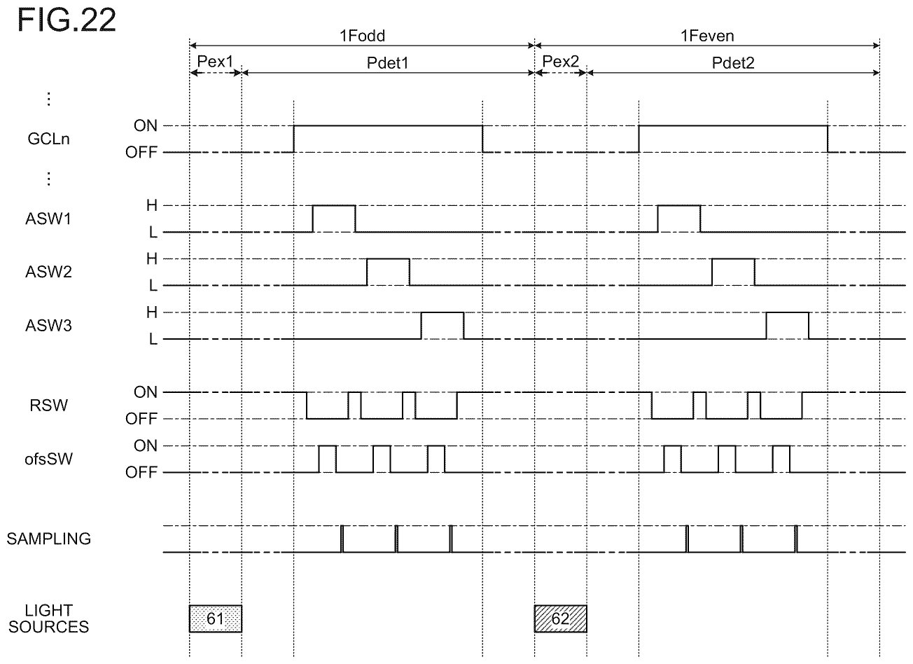

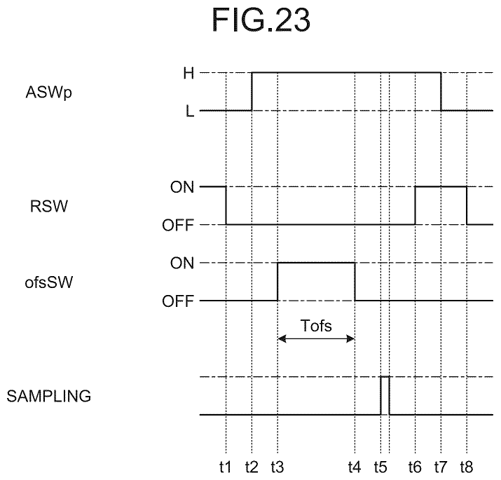

FIG. 22 is a timing waveform diagram illustrating an operation example of the detection device according to the second embodiment. FIG. 23 is an enlarged diagram of the readout period for each of the optical sensors in the timing waveform diagram illustrated in FIG. 22.

Also in the operation example of the detection device 1 according to the second embodiment illustrated in FIGS. 22 and 23, the reset switch RSW is controlled to be on and the reset state is established during the period including the exposure period Pex before time t1. In the operation example of the detection device 1 according to the second embodiment illustrated in FIGS. 22 and 23, the reset switch RSW is controlled to be off and the reset state is terminated at time t1 of the readout period for each of the optical sensors illustrated in FIG. 23. The selection signal ASWp is then controlled to be high (H-controlled) at time t2 after time t1. The offset switch ofsSW is then controlled to be on at time t3 after time t2. The offset switch ofsSW is controlled to be off at time t4 after the offset period Tofs has elapsed. As a result, during the offset period Tofs from time t2 to time t4, the offset current Iofs flows into the negative feedback capacitor Cfb of the detection signal amplifying circuit 42, and the amount of electric charge stored in the negative feedback capacitor Cfb of the detection signal amplifying circuit 42 decreases. The electric charge ΔQofs by which the stored electric charge decreases at this time is Iofs×Tofs (ΔQofs=Iofs×Tofs). As a result, the detection value Raw in the operation example illustrated in FIGS. 22 and 23 is expressed by Expression (18) below.

$$Raw = Vout / ( Vadc / {2}^{n} ) = ( Vref + ( Qphoto - Δ Qofs ) / Cfb \} / ( Vadc / {2}^{n} ) \qquad ( 18 )$$

As a result, in the operation example of the detection device 1 according to the second embodiment illustrated in FIGS. 22 and 23, the detection value Raw is smaller than that in the operation example according to the comparative example for the second embodiment by the offset value ΔRaw expressed by Expression (19) below.

$$Δ Raw = ( Δ Qofs / Cfb ) / ( Vadc / {2}^{n} ) = \{ ( Iofs \times Tofs ) / Cfb \} / ( Vadc / {2}^{n} ) \qquad ( 19 )$$

That is, in the operation example of the detection device 1 according to the second embodiment, the offset value ΔRaw can be set by adjusting the offset current Iofs or the offset period Tofs.

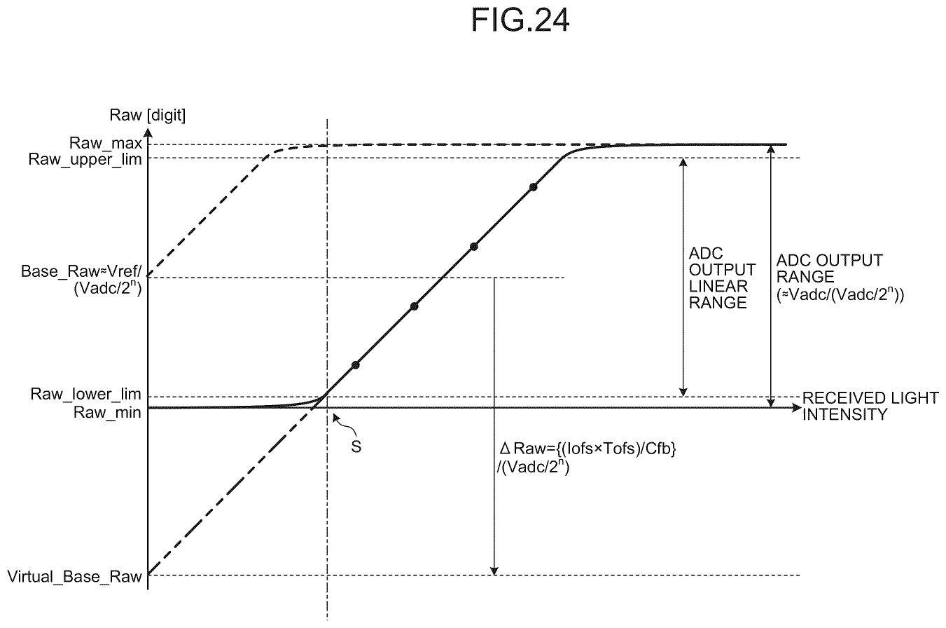

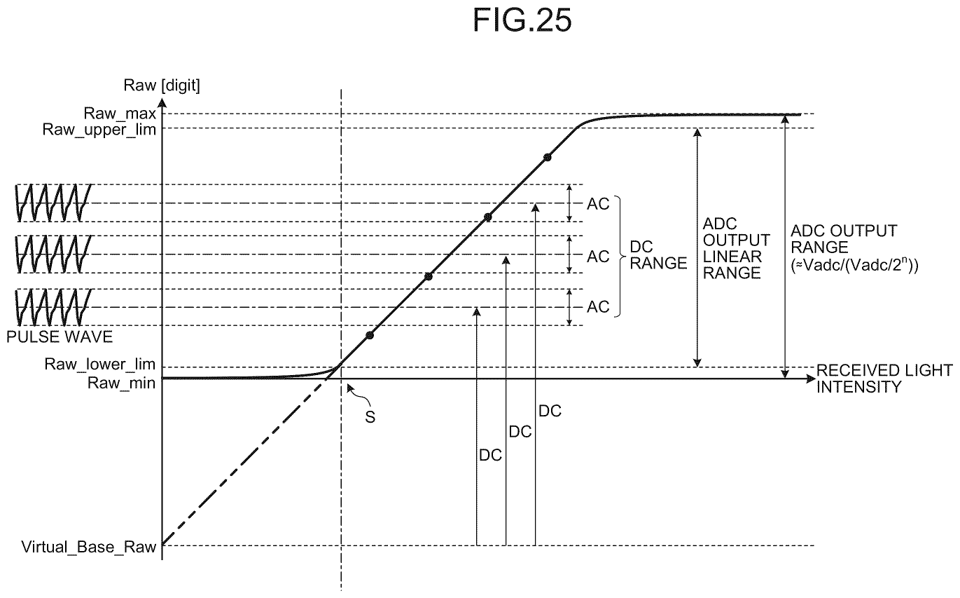

FIG. 24 is a first diagram illustrating the correspondence relation between the detection value of the AFE circuit and the received light intensity of the optical sensor in the operation example of the detection device according to the second embodiment. FIG. 25 is a second diagram illustrating the correspondence relation between the detection value of the AFE circuit and the received light intensity of the optical sensor in the operation example of the detection device according to the second embodiment. In FIGS. 24 and 25, the dashed line illustrates the correspondence relation between the detection value of the AFE circuit and the received light intensity of the optical sensor in the operation example of the detection device according to the comparative example for the second embodiment illustrated in FIG. 21.

The long dashed double-short dashed line illustrated in FIGS. 24 and 25 indicates a hypothetical detection value that is actually limited to the minimum gradation Raw_min of the A/D conversion circuit 43 in a region where the detection value Raw is less than the lower limit gradation Raw_lower_lim.

In the operation example of the detection device 1 according to the second embodiment, as illustrated in FIG. 24, the offset value ΔRaw is larger than that in the operation example of the detection device according to the comparative example for the second embodiment illustrated by a dashed line. Specifically, the offset value ΔRaw is set so as to satisfy Expression (20) or (21) below. As a result, as illustrated in FIG. 25, the lower limit of the range of the detection value Raw used for acquiring the pulse waves can be brought closer to the lower limit gradation Raw_lower_lim, and the range of the detection value Raw used for acquiring the pulse waves can be expanded to the lower limit of the detection range of the detection device 1.

$$Raw\_lower \_lim \geq Vref / ( Vadc / {2}^{n} ) - Δ Raw = ( Vref - \{ ( Iofs \times Tofs ) / Cfb \} ) / ( Vadc / {2}^{n} ) \qquad ( 20 )$$

$$Raw\_lower \_lim \times ( Vadc / {2}^{n} ) \geq Vref - Δ Raw \times ( Vadc / {2}^{n} ) = Vref - \{ ( Iofs \times Tofs ) / Cfb \} \qquad ( 21 )$$

As a result, by increasing the received light intensity during the exposure period Pex, the AC components (AC(Red) and AC(IR), refer to Expression (1) above) of the pulse waves within the detection range can be increased and the detection accuracy of waveforms of the pulse waves can be improved. To increase the received light intensity during the exposure period Pex, for example, the emission intensity of the light sources (first and second light sources 61 and 62) may be increased or the negative feedback capacitor Cfb may be reduced.

In the operation example of the detection device 1 according to the second embodiment illustrated in FIGS. 24 and 25, in a region where the received light intensity of the optical sensor PD during the exposure period Pex is smaller than the predetermined value S, the detection value Raw is non-linear with respect to the variation of the output voltage signal Vout; and as the received light intensity of the optical sensor PD during the exposure period Pex becomes smaller than the predetermined value S, the detection value Raw is limited to the minimum gradation Raw_min of the A/D conversion circuit 43. In other words, in a region where the output voltage signal Vout of the detection signal amplifying circuit 42 is below a predetermined value, the detection value Raw is non-linear with respect to the variation of the output voltage signal Vout; and as the received light intensity of the optical sensor PD during the exposure period Pex becomes smaller than the predetermined value S, the detection value Raw becomes limited to the minimum gradation Raw_min of the A/D conversion circuit 43. That is, in the operation example of the detection device 1 according to the second embodiment, the detection value Raw is limited to the minimum gradation Raw_min (minimum detection value) of the A/D conversion circuit (output circuit) 43 when the light sources (first and second light sources 61 and 62) are off.

The region where the detection value Raw is non-linear beyond the lower limit gradation Raw_lower_lim is a region outside the detection range in the detection device 1. If the detection range of the detection device 1 is assumed to be infinite, the detection value Raw will be linear over the entire range of the output voltage signal Vout. The long dashed double-short dashed line illustrated in FIGS. 24 and 25 indicates a hypothetical detection value when the detection range of the detection device 1 is assumed to be infinite in the region where the detection value Raw is below the lower limit gradation Raw_lower_lim. As described above, the detection value Raw is actually limited to the minimum gradation Raw_min of the A/D conversion circuit 43.

In order to calculate the DC components (DC(Red) and DC(IR), refer to Expression (1) above) of the pulse waves used to calculate the blood oxygen saturation level (SpO₂), the virtual reference value Virtual_Base_Raw needs to be set instead of the reference value Base_Raw. The following describes methods for setting the virtual reference value Virtual_Base_Raw in the operation example of the detection device 1 according to the second embodiment.

### First Method for Setting Virtual Reference Value According to Second Embodiment

As illustrated in FIG. 24, the virtual reference value Virtual_Base_Raw can be calculated by subtracting the offset value ΔRaw (=(ΔQofs/Cfb)/(Vadc/2ⁿ)) from the reference value Base_Raw when the offset period Tofs is set to approximately zero in the operation example of the detection device 1 according to the second embodiment in the state where the optical sensor PD is not exposed to light during the exposure period Pex. In other words, the virtual reference value Virtual_Base_Raw can be calculated by subtracting the offset value ΔRaw from the reference value Base_Raw when the offset period Tofs is set to approximately zero in the operation example of the detection device 1 according to the second embodiment in the state where the optical sensor PD is not exposed to light during the exposure period Pex. In this case, the virtual reference value Virtual_Base_Raw is expressed by Expression (22) below.

$$Virtual\_Base \_Raw = Base\_Raw - Δ Raw = ( Vref - \{ ( Iofs \times Tofs ) / Cfb \} ) / ( Vadc / {2}^{n} ) \qquad ( 22 )$$

### Second Method for Setting Virtual Reference Value According to Second Embodiment

There is variation between the optical sensors PD. Therefore, the reference value Base_Raw is set to a value obtained for each of the optical sensors PD. The reference value Base_Raw is a value obtained when the offset period Tofs is set to approximately zero in the operation example of the detection device 1 according to the second embodiment while the optical sensor PD is not exposed to light during the exposure period Pex. The reference value Base_Raw is, for example, a value obtained and set at shipment or the like of the detection device 1. When Base_Raw(m, n) denotes the reference value of the optical sensor PD in the mth column and nth row, the virtual reference value Virtual_Base_Raw(m, n) for the optical sensor PD in the mth column and nth row is expressed by Expression (23) below.

$$Virtual\_Base \_Raw ( m , n ) = Base\_Raw ( m , n ) - Δ Raw = Base\_Raw ( m , n ) - \{ ( Iofs \times Tofs ) / Cfb \} ) / ( Vadc / {2}^{n} ) \qquad ( 23 )$$

### Third Method for Setting Virtual Reference Value According to Second Embodiment

There are variations in the offset current Iofs, the offset period Tofs, and the negative feedback capacitor Cfb of the detection signal amplifying circuit 42. Therefore, it is preferred to acquire the detection values Raw at multiple points (four points in the example illustrated in FIG. 24) within the detection range (Raw_lower_lim≤Raw≤Raw_upper_lim) illustrated in FIG. 24, and set the virtual reference value Virtual_Base_Raw(m, n) for each of the optical sensors PD, for example, by linear approximation (straight-line approximation) using the least-squares method. The virtual reference value Virtual_Base_Raw(m, n) for each of the optical sensors PD is, for example, a value obtained and set at shipment or the like of the detection device 1. The method for calculating the virtual reference value Virtual_Base_Raw(m, n) for each of the optical sensors PD is not limited to the linear approximation.

In the second embodiment, the signal processing circuit 44 calculates the virtual reference value Virtual_Base_Raw using any one of the first method for setting the virtual reference value according to the second embodiment, the second method for setting the virtual reference value according to the second embodiment, and the third method for setting the virtual reference value according to the second embodiment described above. By storing the virtual reference value Virtual_Base_Raw set in this way in the storage circuit 46, the DC components (DC(Red) and DC(IR), refer to Expression (1) above) of the pulse waves used to calculate the blood oxygen saturation level (SpO₂) can be calculated. As a result, the accuracy of calculation of the blood oxygen saturation level (SpO₂) using the first pulse wave acquired using the first light (red light) and the second pulse wave acquired using the second light (infrared light) can be improved.

### Modification

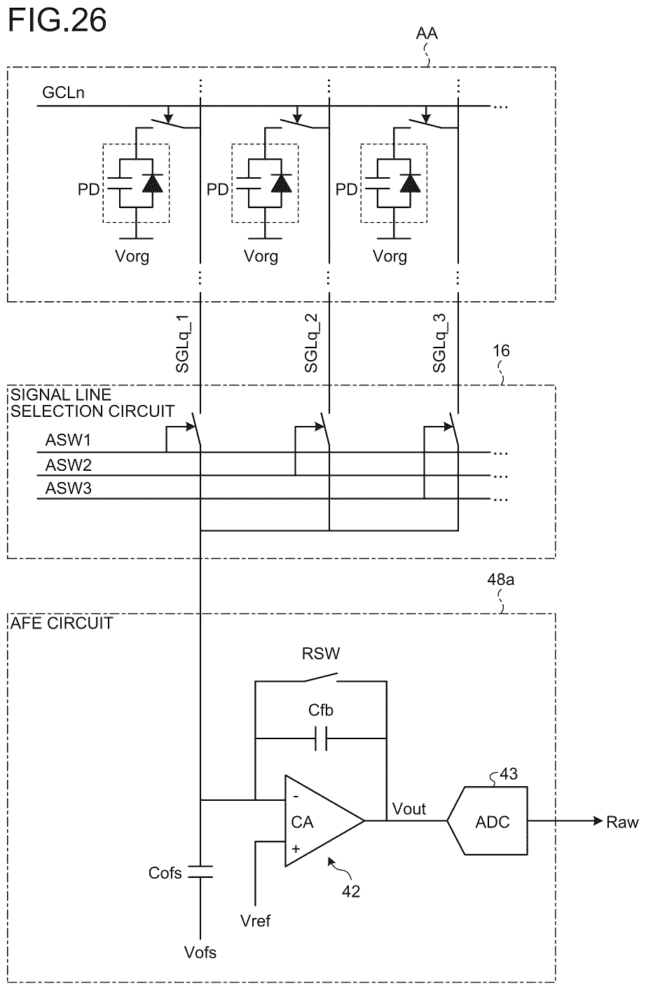

FIG. 26 is a circuit diagram illustrating a configuration example of the AFE circuit according to a modification of the second embodiment. As illustrated in FIG. 26, in the present modification, the inverting input terminal (−) of the differential amplifying circuit CA of the detection signal amplifying circuit 42 included in the AFE circuit 48a receives the offset voltage signal Vofs via the offset capacitor Cofs.

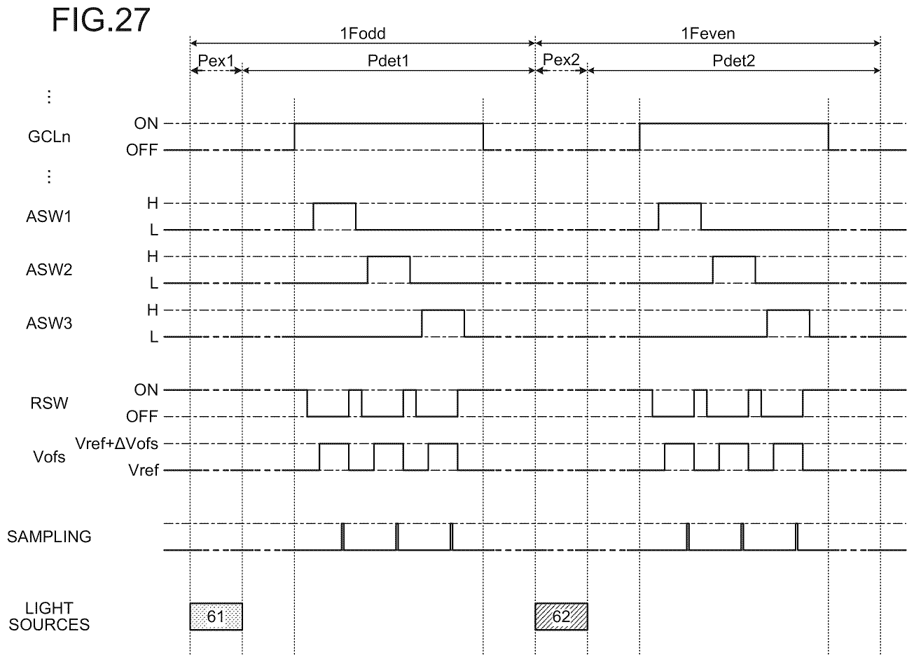

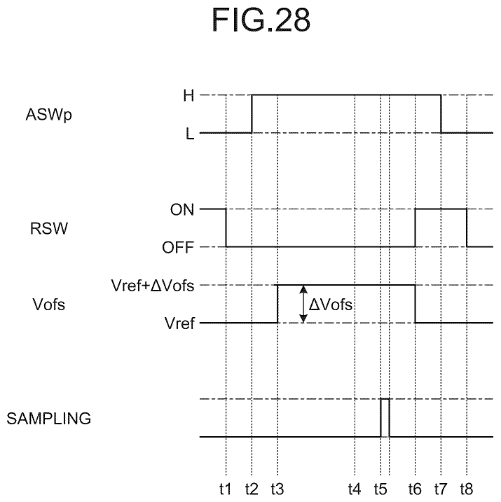

FIG. 27 is a timing waveform diagram illustrating an operation example of the detection device according to the modification of the second embodiment. FIG. 28 is an enlarged diagram of the readout period for each of the optical sensors in the timing waveform diagram illustrated in FIG. 27.

In the operation example of the detection device 1 according to the modification of the second embodiment illustrated in FIGS. 27 and 28, the reference potential Vref is applied as the offset voltage signal Vofs to the offset capacitor Cofs during the period including the exposure period Pex before time t1. As a result, the potential difference between opposite ends of the offset capacitor Cofs is set to approximately zero.

At time t1 of the readout period for each of the optical sensors illustrated in FIG. 28, the reset switch RSW is controlled to be off to terminate the reset state. At time t2 after time t1, the selection signal ASWp is controlled to be high (H-controlled). After time t2, from time t3 to time t6 when the reset switch RSW is controlled to be on, a potential (Vref+ΔVofs) obtained by adding the offset potential ΔVofs to the reference potential Vref is applied to the offset capacitor Cofs. As a result, the offset potential ΔVofs is applied between opposite ends of the offset capacitor Cofs, and a part of the electric charge stored in the capacitive element of the optical sensor PD during the exposure period Pex is transferred to the offset capacitor Cofs. The electric charge ΔQofs transferred at this time is ΔVofs×Cofs (ΔQofs=ΔVofs×Cofs).

As a result, in the operation example of the detection device 1 according to the modification of the second embodiment illustrated in FIGS. 27 and 28, the detection value Raw is smaller than that in the comparative example for the second embodiment by the offset value ΔRaw expressed in Expression (24) below.

$$Δ Raw = ( Δ Qofs / Cfb ) / ( Vadc / {2}^{n} ) = \{ ( Δ Vofs \times Cofs ) / Cfb \} / ( Vadc / {2}^{n} ) \qquad ( 24 )$$

That is, in the operation example of the detection device 1 according to the modification of the second embodiment, the offset value ΔRaw can be set by adjusting the offset potential ΔVofs or the offset capacitor Cofs.

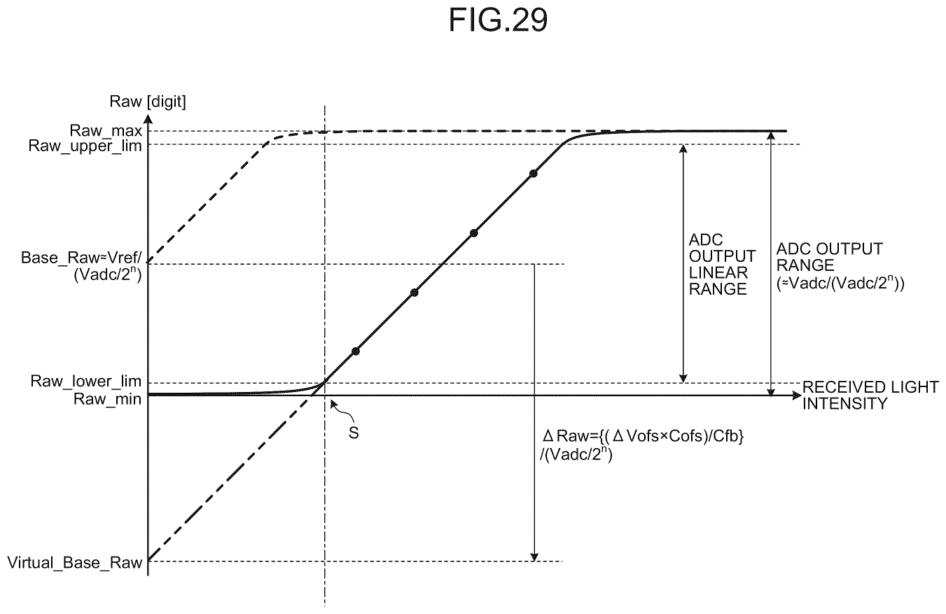

FIG. 29 is a diagram illustrating the correspondence relation between the detection value of the AFE circuit and the received light intensity of the optical sensor in the operation example of the detection device according to the modification of the second embodiment. In FIG. 29, the dashed line illustrates the correspondence relation between the detection value of the AFE circuit and the received light intensity of the optical sensor in the operation example of the detection device according to the comparative example for the second embodiment illustrated in FIG. 21. The long dashed double-short dashed line illustrated in FIG. 29 indicates a hypothetical detection value that is actually limited to the maximum gradation Raw_max of the A/D conversion circuit 43 in the region where the detection value Raw is below the lower limit gradation Raw_lower_lim.

In FIG. 29, the offset value ΔRaw is set so as to satisfy Expression (25) or (26) below. As a result, in the same way as in the operation example of the detection device 1 according to the second embodiment, the lower limit of the range of the detection value Raw used for acquiring the pulse waves can be brought closer to the lower limit gradation Raw_lower_lim, and the range of the detection value Raw used for acquiring the pulse waves can be expanded to the lower limit of the detection range of the detection device 1.

$$Raw\_lower \_lim \geq Vref / ( Vadc / {2}^{n} ) - Δ Raw = ( Vref - \{ ( Δ Vofs \times Cofs ) / Cfb \} ) / ( Vadc / {2}^{n} ) \qquad ( 25 )$$

$$Raw\_lower \_lim \times ( Vadc / {2}^{n} ) \geq Vref - Δ Raw \times ( Vadc / {2}^{n} ) = Vref - \{ ( Δ Vofs \times Cofs ) / Cfb \} \qquad ( 26 )$$

As a result, in the same way as in the operation example of the detection device 1 according to the second embodiment illustrated in FIG. 24, by increasing the received light intensity during the exposure period Pex, the AC components (AC(Red) and AC(IR), refer to Expression (1) above) of the pulse waves within the detection range can be increased and the detection accuracy of waveforms of the pulse waves can be improved. To increase the received light intensity during the exposure period Pex, for example, the emission intensity of the light sources (first and second light sources 61 and 62) may be increased or the negative feedback capacitor Cfb may be reduced.

In the operation example of the detection device 1 according to the modification of the second embodiment illustrated in FIG. 29, in the region where the received light intensity of the optical sensor PD during the exposure period Pex is smaller than the predetermined value S, the detection value Raw is non-linear with respect to the variation of the output voltage signal Vout; and as the received light intensity of the optical sensor PD during the exposure period Pex becomes smaller than the predetermined value S, the detection value Raw is limited to the minimum gradation Raw_min of the A/D conversion circuit 43 in the same way as in the operation example of the detection device 1 according to the second embodiment. In other words, in the region where the output voltage signal Vout of the detection signal amplifying circuit 42 is below the predetermined value, the detection value Raw is non-linear with respect to the variation of the output voltage signal Vout; and as the received light intensity of the optical sensor PD during the exposure period Pex becomes smaller than the predetermined value S, the detection value Raw becomes limited to the minimum gradation Raw_min of the A/D conversion circuit 43 in the same way as in the operation example of the detection device 1 according to the second embodiment. That is, in the detection device 1 according to the modification of the second embodiment, in the same way as in the operation example of the detection device 1 according to the second embodiment, the detection value Raw is limited to the minimum gradation Raw_min (minimum detection value) of the A/D conversion circuit (output circuit) 43 when the light sources (first and second light sources 61 and 62) are off. The region where the detection value Raw is non-linear beyond the lower limit gradation Raw_lower_lim is a region outside the detection range in the detection device 1. If the detection range of the detection device 1 is assumed to be infinite, the detection value Raw will be linear over the entire range of the output voltage signal Vout. The long dashed double-short dashed line illustrated in FIG. 29 indicates a hypothetical detection value when the detection range of the detection device 1 is assumed to be infinite in the region where the detection value Raw is below the lower limit gradation Raw_lower_lim. As described above, the detection value Raw is actually limited to the minimum gradation Raw_min of the A/D conversion circuit 43.

In order to calculate the DC components (DC(Red) and DC(IR), refer to Expression (1) above) of the pulse waves used to calculate the blood oxygen saturation level (SpO₂), the virtual reference value Virtual_Base_Raw needs to be set instead of the reference value Base_Raw. The following describes methods for setting the virtual reference value Virtual_Base_Raw in the operation example of the detection device 1 according to the modification of the second embodiment.

### First Method for Setting Virtual Reference Value According to Modification of Second Embodiment

As illustrated in FIG. 29, the virtual reference value Virtual_Base_Raw can be calculated by subtracting the offset value ΔRaw (=(ΔQofs/Cfb)/(Vadc/2ⁿ)) from the reference value Base_Raw in the operation example of the comparative example for the second embodiment in the state where the optical sensor PD is not exposed to light during the exposure period Pex. In other words, the virtual reference value Virtual_Base_Raw can be calculated by subtracting the offset value ΔRaw from the reference value Base_Raw when the offset potential ΔVofs applied between opposite ends of the offset capacitor Cofs is set to approximately zero in the operation example according to the modification of the second embodiment in the state where the optical sensor PD is not exposed to light during the exposure period Pex. In this case, the virtual reference value Virtual_Base_Raw is expressed by Expression (27) below.

$$Virtual\_Base \_Raw = Base\_Raw - Δ Raw = ( Vref - \{ ( Δ Vofs \times Cofs ) / Cfb \} ) / ( Vadc / {2}^{n} ) \qquad ( 22 )$$

### Second Method for Setting Virtual Reference Value According to Modification of Second Embodiment

There is variation between the optical sensors PD. Therefore, the reference value Base_Raw is set to a value obtained for each of the optical sensors PD. The reference value Base_Raw is a value obtained when the offset potential ΔVofs applied between opposite ends of the offset capacitor Cofs is set to approximately zero in the operation example according to the modification of the second embodiment while the optical sensor PD is not exposed to light during the exposure period Pex. The reference value Base_Raw is, for example, a value obtained and set at shipment or the like of the detection device 1. When Base_Raw(m, n) denotes the reference value of the optical sensor PD in the mth column and nth row, the virtual reference value Virtual_Base_Raw(m, n) for each of the optical sensors PD in the mth column and nth row is expressed by Expression (28) below.

$$Virtual\_Base \_Raw ( m , n ) = Base\_Raw ( m , n ) - Δ Raw = Base\_Raw ( m , n ) - \{ ( Δ Vofs \times Cofs ) / Cfb \} ) / ( Vadc / {2}^{n} ) \qquad ( 23 )$$

### Third Method for Setting Virtual Reference Value According to Modification of Second Embodiment

There are variations in the offset potential ΔVofs, the offset capacitor Cofs, and the negative feedback capacitor Cfb of the detection signal amplifying circuit 42. Therefore, the virtual reference value Virtual_Base_Raw(m, n) is set for each of the optical sensors PD. For example, it is preferred to acquire the detection values Raw at multiple points (four points in the example illustrated in FIG. 29) within the detection range (Raw_lower_lim≤Raw≤Raw_upper_lim) illustrated in FIG. 29, and set the virtual reference value Virtual_Base_Raw(m, n) for each of the optical sensors PD, for example, by the linear approximation (straight-line approximation) using the least-squares method. The virtual reference value Virtual_Base_Raw(m, n) for each of the optical sensors PD is, for example, a value obtained and set at shipment or the like of the detection device 1. The method for calculating the virtual reference value Virtual_Base_Raw(m, n) for each of the optical sensors PD is not limited to the linear approximation.

In the modification of the second embodiment, the signal processing circuit 44 calculates the virtual reference value Virtual_Base_Raw using any one of the first method for setting the virtual reference value according to the modification of the second embodiment, the second method for setting the virtual reference value according to the modification of the second embodiment, and the third method for setting the virtual reference value according to the modification of the second embodiment described above. By storing the virtual reference value Virtual_Base_Raw set in this way in the storage circuit 46, the DC components (DC(Red) and DC(IR), refer to Expression (1) above) of the pulse waves used to calculate the blood oxygen saturation level (SpO₂) can be calculated in the same way as in the second embodiment. As a result, the accuracy of calculation of the blood oxygen saturation level (SpO₂) using the first pulse wave acquired using the first light (red light) and the second pulse wave acquired using the second light (infrared light) can be improved.

In the embodiments described above, the configuration in which the optical sensors PD are arranged in a matrix having a row-column configuration in the detection area AA of the sensor area 10 has been described, but the configuration according to the present disclosure is not limited thereto, and can be applied to a configuration including, for example, one or several optical sensors PD.

While the preferred embodiments have been described above, the present invention is not limited to these embodiments. The content disclosed in the embodiments is merely an example, and can be variously modified within the scope not departing from the gist of the present invention. Any modifications appropriately made within the scope not departing from the gist of the present invention also naturally belong to the technical scope of the present invention. At least one of various omissions, substitutions, and modifications of the components can be made without departing from the gist of the embodiments and the modifications described above.

What is claimed is:

1. A detection device comprising:
an optical sensor;
a light source configured to emit light to the optical sensor;
a detection signal amplifying circuit configured to convert a variation of current supplied from the optical sensor into a variation of voltage; and
an analog-to-digital (A/D) conversion circuit configured to convert an output voltage signal after being converted into the voltage variation into a digital detection value, wherein
the A/D conversion circuit is configured to limit the detection value to a maximum digital gradation value or a minimum digital gradation value when the light source is off.

2. The detection device according to claim 1, wherein
the optical sensor is an organic photodiode and comprises:
an active layer;
an upper electrode provided with an upper buffer layer interposed between the upper electrode and the active layer; and
a lower electrode provided with a lower buffer layer interposed between the lower electrode and the active layer.

3. The detection device according to claim 1, wherein the detection device has
an exposure period during which the light is emitted from the light source to the optical sensor; and
a readout period during which the detection value is acquired based on an electric charge stored in the optical sensor during the exposure period, and
the detection signal amplifying circuit comprises:
a differential amplifying circuit in which a reference potential is applied to a non-inverting input terminal and the optical sensor is coupled to an inverting input terminal; and
a negative feedback capacitor coupled between the inverting input terminal and an output terminal of the differential amplifying circuit.

4. The detection device according to claim 3, wherein the inverting input terminal of the differential amplifying circuit is coupled to an anode of the optical sensor.

5. The detection device according to claim 4, wherein the differential amplifying circuit is provided with, in the readout period, an offset period during which a predetermined offset current flows out of the non-inverting input terminal.

6. The detection device according to claim 5, further comprising a signal processing circuit configured to acquire a pulse wave based on the detection value, wherein
the signal processing circuit is configured to calculate a direct-current (DC) component of the pulse wave based on a preset virtual reference value.

7. The detection device according to claim 6, wherein the signal processing circuit is configured to set the virtual reference value by adding an offset value determined by the offset period and the offset current to a reference value acquired in the readout period in which the offset period is set to approximately zero in a state where the optical sensor is not exposed to light during the exposure period.

8. The detection device according to claim 6, wherein the signal processing circuit is configured to
acquire detection values at multiple points within a detection range in which a detection value that changes linearly with respect to a variation of the output voltage signal is detectable, and
set the virtual reference value based on the detection values at the multiple points.

9. The detection device according to claim 4, wherein
the inverting input terminal of the differential amplifying circuit is configured to receive an offset voltage signal via an offset capacitor, and
an offset potential is applied between opposite ends of the offset capacitor during a predetermined period in the readout period.

10. The detection device according to claim 9, further comprising a signal processing circuit configured to acquire a pulse wave based on the detection value, wherein
the signal processing circuit is configured to calculate a direct-current (DC) component of the pulse wave based on a preset virtual reference value.

11. The detection device according to claim 10, wherein the signal processing circuit is configured to set the virtual reference value by adding an offset value determined by the offset capacitor and the offset potential to a reference value acquired in the readout period in which the offset potential is set to approximately zero in a state where the optical sensor is not exposed to light during the exposure period.

12. The detection device according to claim 10, wherein the signal processing circuit is configured to
acquire detection values at multiple points within a detection range in which a detection value that changes linearly with respect to a variation of the output voltage signal is detectable, and
set the virtual reference value based on the detection values at the multiple points.

13. The detection device according to claim 3, wherein the inverting input terminal of the differential amplifying circuit is coupled to a cathode of the optical sensor.

14. The detection device according to claim 13, wherein the differential amplifying circuit is provided with an offset period during which a predetermined offset current flows into the inverting input terminal in the readout period.

15. The detection device according to claim 14, further comprising a signal processing circuit configured to acquire a pulse wave based on the detection value, wherein
the signal processing circuit is configured to calculate a direct-current (DC) component of the pulse wave based on a preset virtual reference value.

16. The detection device according to claim 15, wherein the signal processing circuit is configured to set the virtual reference value by subtracting an offset value determined by the offset period and the offset current from a reference value acquired in the readout period in which the offset period is set to approximately zero in a state where the optical sensor is not exposed to light during the exposure period.

17. The detection device according to claim 15, wherein the signal processing circuit is configured to
acquire detection values at multiple points within a detection range in which a detection value that changes linearly with respect to a variation of the output voltage signal is detectable, and
set the virtual reference value based on the detection values at the multiple points.

18. The detection device according to claim 13, wherein
the inverting input terminal of the differential amplifying circuit is configured to receive an offset voltage signal via an offset capacitor, and
an offset potential is applied between opposite ends of the offset capacitor during a predetermined period in the readout period.

19. The detection device according to claim 18, further comprising a signal processing circuit configured to acquire a pulse wave based on the detection value, wherein
the signal processing circuit is configured to calculate a direct-current (DC) component of the pulse wave based on a preset virtual reference value.

20. The detection device according to claim 19, wherein the signal processing circuit is configured to set the virtual reference value by subtracting an offset value determined by the offset capacitor and the offset potential from a reference value acquired in the readout period in which the offset potential is set to approximately zero in a state where the optical sensor is not exposed to light during the exposure period.

21. The detection device according to claim 19, wherein the signal processing circuit is configured to
acquire detection values at multiple points within a detection range in which a detection value that changes linearly with respect to a variation of the output voltage signal is detectable, and
set the virtual reference value based on the detection values at the multiple points.

22. The detection device according to claim 4, comprising a plurality of the optical sensors, wherein
the optical sensors are configured to be sequentially coupled to the detection signal amplifying circuit during the readout period.

23. The detection device according to claim 13, comprising a plurality of the optical sensors, wherein
the optical sensors are configured to be sequentially coupled to the detection signal amplifying circuit during the readout period.

24. The detection device according to claim 22, comprising a plurality of the detection signal amplifying circuits and a plurality of the A/D conversion circuits, wherein
a first optical sensor coupled to a first detection signal amplifying circuit and a second optical sensor coupled to a second detection signal amplifying circuit are configured to be simultaneously selected during the readout period.

25. The detection device according to claim 23, comprising a plurality of the detection signal amplifying circuits and a plurality of the A/D conversion circuits, wherein
a first optical sensor coupled to a first detection signal amplifying circuit and a second optical sensor coupled to a second detection signal amplifying circuit are configured to be simultaneously selected during the readout period.

26. The detection device according to claim 24, comprising:
a sensor area in which a plurality of the optical sensors are arranged in a matrix having a row-column configuration in a detection area;
a plurality of gate lines that are each coupled to the optical sensors arranged in a row direction and are arranged in a column direction; and
a gate line drive circuit configured to sequentially select the gate lines during the readout period.

27. The detection device according to claim 25, comprising:
a sensor area in which a plurality of the optical sensors are arranged in a matrix having a row-column configuration in a detection area;
a plurality of gate lines that are each coupled to the optical sensors arranged in a row direction and are arranged in a column direction; and
a gate line drive circuit configured to sequentially select the gate lines during the readout period.

28. A detection device comprising:
an optical sensor;
a light source configured to emit light to the optical sensor;
a detection signal amplifying circuit configured to convert a variation of current supplied from the optical sensor into a variation of voltage; and
an output circuit configured to convert an output voltage signal after being converted into the voltage variation into a detection value, wherein
the output circuit is configured to limit the detection value to a maximum detection value or a minimum detection value when the light source is off.
